(19) United States
(12) Patent Application Publication
(10) Pub. No.: US 2026/0144813 A1
(43) Pub. Date: May 28, 2026

(54) SOLUBLE FIBER COMPOSITIONS TO PREVENT FATTY LIVER AND ALZHEIMER'S

(71) Applicant: CENTRO DE INVESTIGACIÓN Y DE ESTUDIOS AVANZADOS DEL INSTITUTO POLITÉCNICO NACIONAL, Ciudad De Mexico (MX)

(72) Inventors: Carlos Alfredo Silva Ramírez, Tizayuca (MX); Claudia Perez Cruz, Ciudad de Mexico (MX); Jaime García Mena, Ciudad de Mexico (MX)

(21) Appl. No.: 19/401,646

(22) Filed: Nov. 26, 2025

(30) Foreign Application Priority Data
Nov. 27, 2024 (MX) MX/A/2024/014707

(51) Int. Cl. A61K 31/733; A61K 36/88; A61P 1/00; A61P 25/00

(52) U.S. Cl. CPC A61K 31/733; A61K 36/88; A61P 1/00; A61P 25/00

(57) ABSTRACT

The present invention provides a basis for using soluble fiber, particularly compositions comprising Agave fructans, to prevent or treat the accumulation of lipids, particularly ceramides in the liver and brain and lipofuscin in the liver, improving the overall integrity of both organs, with treatment being feasible to prevent fatty liver disease and also prevent Alzheimer's disease (AD). In the present invention, we analyze alterations in lipid metabolism in the liver and ceramide production in the brain of transgenic mice (APP/PS1, TG) for AD. Our results indicate that microvesicular lipid accumulation within hepatocytes is increased in TG mice compared to the control group. Furthermore, in the brains of TG mice, a greater accumulation of ceramides is observed in the somatosensory cortex and entorhinal cortex; in the CA1 region of the hippocampus and in the motor cortex, no significant increase was observed when compared to the control group.

### FIELD OF THE INVENTION

The present invention relates to therapeutic agents or compositions that prevent the accumulation of fat in the liver and brain, with particular benefits for treating Alzheimer's disease and fatty liver disease, particularly compositions comprising soluble fiber, such as agavins.

### BACKGROUND OF THE INVENTION

Alzheimer's disease (AD) is a chronic neurodegenerative disorder and the most common cause of dementia in old age. Dementias, including AD, currently affect more than 50 million people worldwide (Esquerda-Canals et al., 2017; Lane et al., 2018; Scheltens et al., 2021).

Clinically, AD is associated with progressive cognitive decline, resulting in memory loss and impaired reasoning, as well as the onset of problems related to communication skills (Khan et al., 2020; Lane et al., 2018).

The disease affects several brain regions, mainly the cerebral cortex and hippocampus. In the early stages of AD, damage is localized in the frontal and temporal lobes, progressing to regions of the neocortex and hippocampus, but the progression varies between individuals (Masters et al., 2015).

Neurodegeneration associated with AD is characterized by the extracellular formation of amyloid plaques, resulting from the accumulation of the amyloid beta peptide (AB), and by the intracellular aggregation of neurofibrillary tangles, caused by the hyperphosphorylation of Tau protein. These alterations cause the loss of synaptic connections between neurons, leading to their death and ultimately the loss of brain tissue (Khan et al., 2020).

AD can be clinically classified into different phases or stages; in the “preclinical or presymptomatic” phase, patients experience mild memory loss and tissue-level changes in the cerebral cortex and hippocampus. In the “early or light” stage of AD, symptoms are more noticeable in patients, who experience personality, concentration, memory, and disorientation problems that can affect their daily lives. The next stage, “moderate,” is characterized by increased memory loss and language problems due to damage to the cerebral cortex. During the last stage, known as “severe,” the disease spreads throughout the cortex, with a severe accumulation of Aβ plaques and neurofibrillary tangles (Scheltens et al., 2021); Consequently, the patient present severe cognitive damage that prevents the recognizing their family members and being independent. In addition, the patient has difficulty swallowing food and urinating, which can even lead to death (Breijyeh & Karaman, 2020).

AD is classified into two types: familial or early-onset type and sporadic or late-onset type.

Familial AD is characterized by its strong relationship with genetic factors that increase the likelihood of the disease appearing, even at an early age. The most important factors identified in patients with familial AD are mutations in the genes for amyloid precursor protein (APP), presenilin-1 and 2 (PSEN-1 and PSEN-2), which are responsible for the overproduction of the AB peptide. In addition to mutations in the aforementioned genes, the presence of isoform 4 of the apolipoprotein E gene (ApoE4), which is also associated with sporadic AD, has been observed to be involved in the accumulation of AB peptide plaques and vascular damage in brain (Breijyeh & Karaman, 2020; Giau et al., 2015). However, only 5-8% of AD patients are of the familial type (Reitz et al., 2020).

Sporadic AD is the most common type (more than 80% of cases), and symptoms begin to appear on average at the age of 80. The main cause of this type of AD is the inability to remove AB peptide from the brain (Masters et al., 2015), where it is proposed that soluble AB peptide affects synapses by disrupting synaptic functions and initiating signaling cascades related to inflammation, calcium homeostasis, oxidative stress, and the accumulation of neurofibrillary tangles, which together lead to the progression of neurodegeneration (Esquerda-Canals et al., 2017). Unlike the familial type, most cases of sporadic Alzheimer's disease are not related to specific mutations characteristic of the disease, but rather to multiple risk factors that increase the likelihood of developing the disease. The most common risk factors are metabolic diseases such as obesity, diabetes, and dyslipidemia, cardiovascular diseases, and hypertension (Mayeux & Stern, 2012; Silva et al., 2019).

As mentioned above, there are two neuropathological features associated with AD: the accumulation of neurofibrillary tangles and Aβ peptide plaques (amyloid plaques) (Scheltens et al., 2021). Another notable type of neuropathology in patients is the loss of neuronal processes such as synapses and even brain tissue itself, leading to atrophy. Damage from oxidative stress and neuroinflammation is also observed, with all of these neuropathological features being related to disease progression and the onset of cognitive symptoms in patients (Breijyeh & Karaman, 2020).

Amyloid plaques, also known as senile plaques, are extracellular deposits of the Aβ peptide. These peptides are generated by the proteolytic enzymes β- and γ-secretases from transmembrane APP, producing the final forms of the Aβ peptide: Aβ40 and Aβ42 (Breijyeh & Karaman, 2020; Chen et al., 2017). Multiple mutations have been found in the APP gene, most of which are related to AD. For example, KM670/671NL, located at the site of recognition by proteolytic enzymes, increases the accumulation of amyloid plaques in the hippocampus and cerebral cortex (Li et al., 2019).

Mutations in the PSEN-1 gene are common and are linked to AD because PSEN-1 is a protein responsible for activating the γ-secretase complex and altering Aβ40 and Aβ42 levels. On the other hand, mutations in the PSEN-2 gene are not as common, but they have a severe effect on the production of Aβ40 and Aβ42, causing familial AD even in patients without mutations in the PSEN-1 gene (Cai et al., 2015; Kelleher & Shen, 2017).

The loss of synaptic processes is a very marked neuropathological feature in the early stages of AD, as it compromises regions of the brain, such as the neocortex, related to memory loss.

Systemic alterations and their relationship to Alzheimer's disease. The multifactorial etiology of AD includes medical conditions outside the central nervous system (CNS) as risk factors that increase the likelihood of developing the disease. Hypercholesterolemia, obesity, non-alcoholic fatty liver disease (NAFLD), hyperglycemia, insulin resistance, and hypertension are conditions that are part of a metabolic syndrome, which is related to type 2 diabetes, dementia, neurodegeneration, and an elevated risk of developing AD (Baker et al., 2011; Li et al., 2020).

Multiple studies have shown that obesity increases the likelihood of developing AD in adulthood and middle age (Kivipelto et al., 2018). Patients with obesity have reduced hippocampal volume, as well as a high rate of brain atrophy and poor performance on tasks the memory is evaluated (Coppin et al., 2014; Dye et al., 2017). Several studies in animal models have reported that a high-fat diet to induce obesity is associated with increased accumulation of Aβ and hyperphosphorylated tau, as well as reduced neurogenesis in the hippocampus and problems in performing tasks to assess cognition (Graham et al., 2016; Haleem & Mahmood, 2021; Koga et al., 2014). Weight loss has been reported to be associated with improved cognitive function in obese patients (Espeland et al., 2018).

It has been reported in transgenic mouse models for AD that the induction of diabetes through a high-fat diet has a positive effect on Aβ40 and Aβ42 amyloidogenesis and tau hyperphosphorylation, in addition to memory impairments (Ho et al., 2004; Mehla et al., 2014; Yang et al., 2013).

NAFLD consists of fat accumulation and immune cell infiltration in liver tissue, where the progression of liver disease is accompanied by the release of proinflammatory cytokines (Luo & Lin, 2021). A diet high in cholesterol, saturated fatty acids, and sugars has been shown to have harmful effects on the liver, leading to the accumulation of lipid droplets in hepatocytes and the release of proinflammatory cytokines, thus contributing to the development of NAFLD and systemic inflammation (Pilling et al., 2021). NAFLD induces alterations in lipid homeostasis in the system, attenuating the ability of hepatocytes to eliminate circulating Aβ peptide, thus increasing Aβ accumulation in the brain and the likelihood of developing AD (Bassendine et al., 2020; Gali et al., 2019). In transgenic mouse models for AD, it has been observed that stimulation of Aβ clearance mediated by lipoprotein receptor-bound peptide 1 (LRP-1) in the liver reduces the peptide accumulationin brain and it improves cognitive abilities (Cheng et al., 2020; Sehgal et al., 2012).

These data highlight the importance of good liver function and the progression of neurodegenerative disease, as liver damage reduces peripheral Aβ clearance and increases the release of proinflammatory cytokines, which affect the integrity of the blood-brain barrier and cause systemic and central inflammation (Więckowska-Gacek et al., 2021), events associated with neurodegeneration.

Liver-brain axis. The evident relationship between the liver and the brain has opened up a field of study called the liver-brain axis, which establishes that chronic liver diseases, including NAFLD and alcoholic liver disease (ALD), affect energy homeostasis and the metabolism of toxic compounds that could be involved in the development of neurodegenerative disorders, such as AD and Parkinson's disease (PD).

Liver dysfunction and markers of AD pathology: It has been observed that the overproduction of ammonia in the liver generates free radicals, which affects the performance and signaling of brain proteins through post-translational modifications (Oja et al., 2017; Vegas-Suarez et al., 2022). As mentioned, above, NAFLD decreases the ability to eliminate Aβ peptide, contributing to its accumulation and increasing the likelihood of developing AD (Bassendine et al., 2020). Most liver diseases result in the release of proinflammatory cytokines, causing systemic inflammation and neuroinflammation. Bile acid synthesis in the liver has been linked to neurological disorders because certain alterations in bile acid homeostasis and intestinal microbiota contribute to systemic inflammation and peripheral clearance of Aβ (Vegas-Suárez et al., 2022).

It is known that mitochondrial dysfunction and the generation of oxidative stress can cause alterations in multiple physiological processes in the liver that lead to NAFLD (Masarone et al., 2018). one of which is the accumulation of ceramides and other lipotoxic intermediates due to various problems with beta oxidation (Hensley et al., 2000).

In recent years, it has been proposed that communication between the liver and the brain occurs through the accumulation of sphingolipids called ceramides, as it has been observed that this type of lipid, found in the peripheral blood of rats with steatohepatitis, can cause neurotoxic lesions (Lyn-Cook et al., 2009).

Ceramides: Ceramides are sphingolipids composed of a fatty acid of variable chain length and an amino group (sphingosine). The fatty acid chains that make up ceramides can be saturated or monounsaturated and may have an OH group attached to carbon 2 or the terminal carbon. In mammalian cells, there are short-chain, long-chain (16 to 20 carbons), or very long-chain (22 to 24 carbons) ceramides.

Ceramide biosynthesis can occur in three ways: de novo, hydrolysis or degradation of sphingomyelin, and recycling of sphingolipids (FIG. 1). De novo synthesis occurs in the endoplasmic reticulum using ceramide synthases and serine palmitoyl transferase.

In the catabolic pathway, ceramides are synthesized through the hydrolysis of sphingomyelin by sphingomyelinases. In sphingolipid recycling, the reacylation of sphingosine yields ceramide (Field et al., 2020; Jazvinšćak Jembrek et al., 2015).

Clinical studies have shown that ceramides in the circulation originate mainly in the liver, which is considered an important site for de novo ceramide synthesis (Boon et al., 2013).

The highly hydrophobic nature of ceramides allows them to form part of the cell membrane structure, where they can induce alterations related to vesicle fusion and changes in membrane thickness. In addition, thanks to their hydrophobicity, ceramides can interact with multiple signaling molecules from the membrane, influencing the affinity and activity of enzymes and other proteins (Castro et al., 2014; Tomassini & Testi, 2002). Ceramides are also a fundamental part of the structural integrity of the mitochondrial membrane, where they also participate in apoptosis mediated by damage to the mitochondrial respiratory chain, where this organelle is capable of detecting cellular stress induced by specific lipid species, including ceramides (Ogretmen, 2017; Teixeira et al., 2015; Tomassini & Testi, 2002).

Ceramides are capable of organizing themselves in the cell membrane and forming platforms to aid in signal transduction through the formation of receptor clusters, the recruitment of molecules that interact with receptors, and the stabilization of signaling processes (Castro et al., 2014). It has been proposed that ceramides may affect membrane permeability through the formation of well-structured channels composed of ceramides, allowing the release of ions, proteins, and other molecules (Colombini, 2010). Thus, the overproduction or accumulation of ceramides has been associated with cell damage through multiple mechanisms.

Ceramides have been observed to be involved in various pathological processes in different metabolic diseases, such as diabetes. In recent years, a relationship has been found between insulin resistance, an important factor in the development of type 2 diabetes, and the production of ceramides (Chavez & Summers, 2012), This is because it has been suggested that ceramides are involved in multiple molecular mechanisms of insulin resistance, such as B-cell apoptosis and inflammation in the pancreas, oxidative and mitochondrial stress, and insulin synthesis and release (Galadari et al., 2013).

It is known that various metabolic diseases, such as diabetes, insulin resistance, and hepatic steatosis, originate from excessive fat accumulation in the body, which results in increased blood lipid and cholesterol concentrations, as well as an increase in the availability of free fatty acids, which together raise the amount of lipid metabolites, such as triacylglycerols, diacylglycerols, and ceramides, lipid species related to the progression of the aforementioned metabolic diseases (Aburasayn et al., 2016).

Several studies have reported that ceramide levels in plasma, adipose tissue, and skeletal muscle are elevated in patients with obesity and type 2 diabetes. In addition, high ceramide levels have been found to correlate negatively with insulin sensitivity, indicating a state of insulin resistance, in patients with type 2 diabetes and in animal models. The results are supported by evidence that ceramides reduce the activity of protein kinase B, which is an effector in insulin signaling, as well as apoptosis and cell proliferation (Haus et al., 2009; H. Zhou et al., 1998). In addition, it has been observed that ceramide levels from the adipose tissue of patients with high levels of lipid accumulation in the liver are higher than in patients with healthy livers (Kolak et al., 2007).

NAFLD consists of a diverse range of liver disorders, ranging from the accumulation of fat in the form of droplets in the parenchyma (steatosis) to inflammation and cell damage, a condition known as steatohepatitis, which can progress to the onset of fibrosis, cirrhosis, and subsequently hepatocellular carcinoma. NAFLD is a disease with multiple pathogenesis, where the presence of different comorbidities such as insulin resistance and diabetes, in addition to the overproduction and accumulation of free fatty acids, are related to the onset and progression of this disease (Friedman et al., 2018).

It is known that the accumulation of free fatty acids increases the production of triglycerides, which is classified as a compensatory mechanism when the presence of free fatty acids exceeds the capacity to metabolize them (Bril et al., 2017). When fatty acids are found in excess in the liver, or the ability to process them is abnormal, lipid droplets form in hepatocytes, which can serve as a substrate for the generation of toxic lipid species (Friedman et al., 2018); with diacylglycerols and ceramides being the most relevant in NAFLD (Luukkonen et al., 2016).

It has been detailed that ceramides are directly related to the accumulation of fat in the liver. Likewise, steatosis is also related to the overproduction of ceramides, where the liver acts as a reservoir of ceramides that are constantly released into the circulation; Furthermore, it has been observed that ceramides are related to the progression of steatosis to non-alcoholic steatohepatitis as a result of increased oxidative stress and endoplasmic reticulum stress, cell death, and the activation of hepatic stellate cells (Hajduch et al., 2021).

Ceramides affect lipid metabolism through various mechanisms. Ceramides are known to increase the availability of fatty acids for triglyceride synthesis by increasing the expression of the fatty acid transporter CD36. Another mechanism is described through the modulation of lipogenic gene expression mediated by the transcription factor SREBP-1c (sterol regulatory element binding protein-1c), as it has been observed that, in models of hepatic steatosis and in the liver of patients with NAFLD, the expression of the transcription factor and its related genes is elevated. ceramides also alter lipid metabolism by reducing fatty acid oxidation in the mitochondria (Auguet et al., 2014; Kammoun et al., 2009).

Alterations in lipid metabolism have been identified as an important risk factor for the development and progression of AD. It is known that the presence of the ApoE4 allele is an important genetic risk factor for the development of AD. The main function of the ApoE protein is to transport cholesterol within cells, where different polymorphisms modulate its activity, with the E4 polymorphism having the lowest capacity and the E2 polymorphism having the highest capacity (Liu et al., 2013). The E4 allele is associated with increased amyloid pathology and cognitive decline in carrier patients through the regulation of Aβ peptide accumulation and other processes such as neuroinflammation and lipid transport in the brain (Liu et al., 2013; Mahley & Rall, 2000).

In recent years, mechanisms related to the presence of ceramides that could be involved in the neuropathology of AD have been proposed. In a quintuple transgenic mouse model (5XFAD) model for AD, higher levels of Cer18 (ceramide with an 18-carbon chain) were observed in serum compared to wild-type (WT) mice, with this accumulation increasing with age, peaking at 10 weeks of age (Dinkins et al., 2015). In the same study, it was observed that exogenous administration of Cer18 is related to an increase in amyloid plaques in the brains of female mice (Dinkins et al., 2015). In another study conducted with 3- and 5-month-old 5XFAD mice carrying the ApoE4 allele, it was reported that ceramide levels (Cer14, Cer16, Cer18, Cer20, Cer22, and Cer24) were higher in the cerebral cortex compared to control mice. it was also observed that in female mice, ceramide levels are lower in the hippocampus than in the cortex, compared to male mice (den Hoedt et al., 2021).

The excessive accumulation of ceramides in the brain is relevant since ceramides are known to be involved in the aggregation of the Aβ peptide. There is evidence showing a relationship between ceramide generation, Aβ synthesis, and neuronal death (Cutler et al., 2004). Aβ synthesis usually occurs in lipid rafts, where ceramides can act as stabilizers of the β-secretase enzyme, which is responsible for the synthesis of the Aβ peptide from APP, thereby facilitating the production of Aβ (Puglielli et al., 2003).

Furthermore, some evidence indicates that sphingomyelin metabolism is altered in patients with AD (Baloni et al., 2022), bearing in mind that sphingomyelin hydrolysis is an important pathway for its synthesis, representing a significant source of ceramide production (Cutler et al., 2004). On the other hand, high levels of Aβ induce the activation of sphingomyelinases, causing an increase in the amount of ceramides (Baloni et al., 2022; Tanabe et al., 2013).

Filippov and colleagues observed elevated levels of different ceramide species in the brains of patients diagnosed with AD; the ceramide species they found were Cer16, Cer18, Cer20, and Cer24. Furthermore, the results of the research showed that the regulation of ceramide biosynthesis is dysfunctional in conditions of neurodegeneration (Filippov et al., 2012).

These data show us that there is an alteration in ceramide levels in brains of patients with AD. However, the origin of this accumulation of ceramides in the brain has not been clarified; whether it is of in situ origin or comes from the periphery. In recent years, it has been proposed that ceramides originating in the liver and released into the circulation are capable of crossing the blood-brain barrier and exerting neurotoxic effects, causing neuroinflammation, and inducing alterations in lipid homeostasis, as well as causing damage to mitochondrial function and insulin resistance in the brain (De La Monte & Tong, 2014). One study reported that cognitive impairment in 3-month-old APP/PS1 mice correlates with elevated serum levels of ceramides and tau (Tong, 2014). Tong, 2014). One study reported that cognitive impairment in 3-month-old APP/PS1 mice correlates with elevated serum levels of ceramides and phosphorylated tau. In addition, they observed that different genes related to ceramide synthesis are found at elevated levels in the liver, an organ where they also reported increases in proinflammatory cytokines and free fatty acids (Liu et al., 2024). Thanks to this information, it is relevant to study ceramides not only in the brain but also in the liver, providing valuable knowledge in understanding the etiology of AD.

Intestinal microbiota: The intestinal microbiota in humans is composed of approximately 10¹³ microorganisms that reside in the intestine, where bacteria belonging to the Bacteroidetes and Firmicutes families predominate (Jandhyala et al., 2015). It is known that, in murine models, disturbances in the composition of the intestinal microbiota in early life can lead the host to develop metabolic conditions such as obesity and diabetes (Cox et al., 2014). The term dysbiosis refers to changes in the composition and abundance of the intestinal microbiota due to various factors, such as diet, gastric pH, and intestinal motility, as well as environmental and immunological factors (Leung et al., 2016). These changes in the composition of the microbiota can have various physiological and pathological effects on the host.

The intestinal microbiota can modulate various physiological processes in the host through metabolites generated as a result of the fermentation of nutrients from the diet, which are absorbed by the intestine and carried into the blood and lymphatic system (Wang et al., 2019). Metabolites, such as short-chain fatty acids and trimethylamine, have been studied for their effects on multiple processes, such as maintaining energy homeostasis in the host (Kasubuchi et al., 2015) and activating the inflammatory response (Koeth et al., 2013), respectively. Another very important process carried out by the gut microbiota is its involvement in bile acid metabolism. Different bacterial genera, such as Lactobacilli, Clostridium, and Bacteroides, are capable of producing an enzyme called bile salt hydrolase (BSH), whose main function is to break down primary bile acids in the small intestine (Wang et al., 2019). The BSH enzyme has been studied for its role in nutrition, in reducing bile salt toxicity, and in lipid homeostasis in the host (Chand et al., 2017).

In recent years, it has been reported that the intestinal microbiota is related to the onset and progression of various metabolic diseases.

In studies with animal models and humans, dysbiosis has been observed to be related to the pathogenesis of obesity. For example, it has been reported that the intestinal microbiota of obese mice has a high capacity to generate energy (Turnbaugh et al., 2006). It has also been observed that, in obese individuals, the abundance of Bacteroides is attenuated, which is negatively related to resistance to fat accumulation (Liu et al., 2017). Another mechanism by which dysbiosis could act on the development of metabolic diseases is the loss of intestinal integrity. It has been reported that a high-fat diet generates dysbiosis and elevated production of metabolites that affect the integrity of tight junctions in intestinal tissue, increasing permeability to various microbial metabolites that can be transported into the circulation and exert their effects on conditions related to metabolic syndrome (Dabke et al., 2019).

In relation to NAFLD, various mechanisms focused on the pathogenesis linked to dysbiosis have been studied, from the participation of short-chain fatty acids to the activation of the farnesoid X receptor (FXR) in the intestine, the latter being highly relevant, as it has been shown that changes in the gut microbiota are capable of altering the composition of bile acids, resulting in antagonistic activity of the FXR receptor, leading to lipogenesis and ceramide synthesis in the liver (Jiang et al., 2015). In the intestine, the primary bile salt called tauro-beta-mucarolic acid (T-β-MCA) is secreted by the liver to inhibit FXR activity and mediate the accumulation of lipids in the liver (Wang et al., 2019). Other studies have reported that dysbiosis caused by a high-fat diet induces the proliferation of bacterial species related to BSH production and T-β-MCA hydrolysis, thereby reducing FXR inhibitor concentrations, which translates into increased receptor activity in the intestine (Li et al., 2013; Wang et al., 2019). Alterations in lipid metabolism caused by FXR activation in the intestine have a direct effect on triglyceride metabolism in the liver. This communication occurs through FXR-mediated ceramide synthesis in the intestine. Inhibition of FXR signaling is associated with a reduction in serum ceramide levels, which also reduces the activity of the transcription factor SREBP-1c, involved in free fatty acid synthesis, thereby decreasing lipid accumulation in the liver (Jiang et al., 2015; Wang et al., 2019).

In addition to the effects of the intestinal microbiota on systemic processes, there is evidence of its involvement in various CNS pathologies, with AD being one of the most studied. Currently, the microbiota-intestine-brain axis has been proposed with the aim of studying the association of the intestinal microbiota with neurodegenerative diseases, particularly AD (Liu et al., 2020).

Remarkable alterations have been observed in various analyses of the composition and diversity of the gut microbiota in patients with AD and in animal models of AD. In a study of patients with AD, it was observed that the intestinal microbiota is characterized by a decrease in the diversity and abundance of its composition compared to healthy volunteers of the same age and sex but without a diagnosis of dementia (Vogt et al., 2017). The same study observed a reduction in the phylum Firmicutes in patients with AD, a characteristic that has been observed in studies of the gut microbiota in metabolic diseases such as obesity and diabetes, which are associated with an increased risk of developing AD (De La Monte & Wands, 2005). On the other hand, the study observed that the intestinal microbiota of patients with AD showed an increase in the phylum Bacteroidetes, which is composed of a large group of Gram-negative bacteria capable of producing lipopolysaccharides (LPS) that can cross the intestinal barrier into the circulation and thus cause an inflammatory response in the host (Asti & Gioglio, 2014). Gioglio, 2014). Different studies have demonstrated the relationship between LPS and various AD pathologies, such as their involvement in amyloid fibrillogenesis (Asti & Gioglio, 2014) and in tau protein accumulation (Kitazawa et al., 2005).

With regard to animal models of AD, one study reported that 8-month-old APP/PS1 transgenic mice show a significant increase in Bacteroidetes and a reduction in Firmicutes in the composition of the intestinal microbiota, compared to wild-type mice of the same age that do not express the mutation (Harach et al., 2017). In addition, it was observed that the accumulation of Aβ peptide in the brain is reduced in APP/PS1 transgenic mice lacking intestinal microbiota, indicating that specific species of microorganisms could be related to the pathology generated by the Aβ peptide (Harach et al., 2017).

As mentioned above, in patients with AD and in murine models of the disease, it has been reported that intestinal dysbiosis is related to the disease. Because, it has been proposed that modulating the intestinal microbiota may modulate the course of AD. In a previous study by our research group, we evaluated the impact of a dietary portfolio on cognitive functions and the intestinal microbiota of a triple transgenic mouse model (3×TGAD). The intake of bioactive foods significantly improves cognitive function and reduces neuroinflammation, all of which is associated with changes in the gut microbiota (Syeda et al., 2018).

The role of diet in liver function and AD prevention: Currently, lifestyle modifications, such as physical exercise or a healthy diet, have been shown to have a positive impact on the development and treatment of certain diseases, including NAFLD and AD (Romero-Gómez et al., 2017; Shah, 2013).

Diet composition is known to be involved in the development of NAFLD. On the one hand, the consumption of added sugars, such as high-fructose corn syrup present in processed foods and beverages, has been associated with increased triglyceride synthesis in liver (Angelopoulos et al., 2009). In addition, fructose metabolism has been linked to changes in the intestinal microbiota, increased intestinal permeability, production of proinflammatory cytokines in the liver, and hepatic steatosis (Bergheim et al., 2008). On the other hand, it has been proposed that the Mediterranean diet, characterized by reduced sugar consumption and increased consumption of monounsaturated fats, fish, fruits, and vegetables, plays an important role in reducing the risk of cardiovascular diseases, such as type 2 diabetes mellitus, related to NAFLD (Salas-Salvadó et al., 2014).

Furthermore, a study of patients with NAFLD has suggested that following a Mediterranean diet is associated with a reduction in hepatic fat accumulation (Trovato et al., 2015).

The Mediterranean diet is also associated with protection against the development of dementia and AD. Several studies and meta-analyses have shown an association between following a Mediterranean diet and a reduced risk of developing AD and improved brain health (Feart et al., 2015; Singh et al., 2014).

It has been proposed that the mechanism by which the Mediterranean diet may exert its effects on the development of AD is due to the soluble fiber content present in green leafy vegetables, garlic, and onions. Once consumed, soluble fiber acts as a substrate for microorganisms in the intestine, promoting the survival and selective proliferation of bacterial species that have been linked to the production of metabolites (such as short-chain fatty acids) with health benefits (Guan et al., 2021).

Fructans are a type of soluble fiber found in a wide variety of foods, such as fruits, vegetables, and tubers, and other plant sources such as the stem of the blue variety of Agave tequilana (Mellado-Mojica et al., 2017), which have been associated with improved metabolic conditions such as weight reduction, fat tissue volume, and cholesterol levels in obese mice (Márquez-Aguirre et al., 2013).

It has been reported that, in obese patients, the intake of Agave fructans reduced body mass index, total body fat, and triglyceride levels, without causing any aversion or side effects (Padilla-Camberos et al., 2018).

It has been reported that the diet in TG mice modifies the intestinal microbiota and brain function. In a first study, a 9-month-old 3×TGAD (transgenic Alzheimer's disease) model was used, fed a set of bioactive foods for 7 months. It was observed that 3×TGAD mice treated with bioactive foods showed cognitive improvements and reduced neuroinflammation, both events associated with changes in the intestinal microbiota and LPS production (Syeda et al., 2018).

Problem statement: AD has a multifactorial etiology, with alterations in peripheral organs, and systemic diseases having been described as risk factors for developing the pathology. It has been proposed that alterations in lipid metabolism in the liver lead to the accumulation of triglycerides in hepatocytes (a condition called steatosis), generating lipotoxic species and affecting liver function and other organs, including the brain. In conditions of steatosis in the liver, excess ceramides are produced, which can be released into the circulation where they have the capacity to exert negative effects on distant organs, such as the brain.

Furthermore, it is known that intestinal dysbiosis, a disorder present in patients with AD, is a trigger for hepatic steatosis, where an increase in the amount of certain bacterial populations and their metabolites can activate signaling pathways in the intestine related to ceramide synthesis, which can reach the liver and activate transcription factors involved in hepatic steatosis. Furthermore, it has been shown that the inclusion of soluble fiber in the diet has positive effects on various peripheral diseases, and even at the central level, due to the modulation of the intestinal microbiota and the generation of short-chain fatty acids, such as butyrate, capable of improving liver and brain function.

The present invention determines the behavior of lipid accumulation in the liver and ceramides in the brain of transgenic mice for Alzheimer's disease and demonstrates that intervention with soluble fiber can prevent liver alterations.

The following abbreviations were used for the present invention: ACh Acetylcholine, ALT Alanine aminotransferase, ApoE Apolipoprotein E, APP Amyloid precursor protein, BSA Bovine serum albumin, BSH Bile salt hydrolase, AD Alzheimer's disease, PD Parkinson's disease, FXR Farnesoid X receptor, GLP-1 Glucagon-like peptide-1, LPS Lipopolysaccharides, LRP-1 Lipoprotein receptor-related protein 1, NaBH4 Sodium borohydride, NAFLD Non-alcoholic fatty liver disease, PCR Polymerase chain reaction, PFA Paraformaldehyde, PSEN Presenilin, SREBP-1c Sterol regulatory element-binding protein-1c, T-β-MCA Muracolic-tauro-beta acid.

### BRIEF DESCRIPTION OF THE FIGURES

FIG. 1. Ceramide synthesis. Ceramide synthesis can occur in three ways: de novo synthesis, sphingolipid recycling, and sphingomyelin hydrolysis.

FIG. 2. Relative abundance of taxa in fecal samples from WT+C, TG+C, TG+F, and TG+F mice treated with antibiotics (TG+F+Abx). A. Phyla. B. Taxa. The abundance of Lactobacillus is indicated in brackets.

FIG. 3. Levels of short-chain fatty acids in feces from WT and TG mice treated with a control diet (C) or fiber (F).

FIG. 4. Intestinal morphology of WT and TG mice treated with diet C or F. A clear alteration is shown in TG mice, which was reduced after fructan intake. Arrow: submucosa. Arrowhead: Muscular layer. Scale bar: 100 μm.

FIG. 5. Oil red staining. The staining identifies neutral lipids within the cell. The specific dye mark is shown in the form of red droplets indicated by the yellow arrows.

FIG. 6. Representation of color threshold adjustment with ImageJ for quantification of the specific mark. A. The area of the specific mark is seen in black due to the color threshold adjustment (yellow arrows). B. ImageJ program interface for adjusting hue, saturation, and brightness values.

FIG. 7. Sudan Black B staining. The stain identifies lipofuscin within the cell. The dye mark is seen as intense blue in the perinuclear area, indicated by the yellow arrows.

FIG. 8. Color threshold adjustment with ImageJ for quantification of the specific Sudan Black B dye mark. The specific mark area appears black due to the color threshold adjustment (yellow arrows). B. ImageJ program interface for adjusting hue, saturation, and brightness values.

FIG. 9. Representative image of the right hemisphere of the mouse brain with the analyzed regions marked. The CA1 region of the hippocampus and the entorhinal, somatosensory, and motor cortices are marked.

FIG. 10. Oil Red O staining in the livers of TG and WT mice. Representative images of the staining in liver tissue. WT+Chow mice show positive Oil Red O staining, but the levels are low. The stain is red and drop-shaped. B. TG+Chow mice show abundant lipid microvesicles within hepatocyte, surrounding the cell nucleus (blue). C. Semi-quantitative results of the percentage of area stained with Oil Red O in the study groups, where TG+Chow showed higher levels compared to WT-Chow mice. Data are shown as mean±SD. Statistical analysis performed with the unpaired Student's t-test; ****p<0.0001. Scale bar: 50 μm.

FIG. 11. Accumulation of ceramides in the brain determined by immunofluorescence. Representative images of ceramide immunoreactivity (ceramides+ir, green) in the CA1 region of WT+Chow and TG+Chow mice. Cell nuclei in blue. I-L. Quantitative analysis of ceramide+ir by brain region (□m⁽²⁾). Data are shown as mean±SD. Statistical analysis performed using the unpaired Student's t-test; ns=no significant difference *p<0.05. Scale bar: 50 μm.

FIG. 12. Quantification of ceramide levels (ceramide+ir) in all brain regions analyzed in WT+Chow and TG+Chow mice. Data are shown as mean±SD. Statistical analysis performed with the unpaired Student's t-test; ***p<0.001.

FIG. 13. Oil red O staining in liver tissue from WT and TG mice fed a control diet (C) or fiber diet (F). Representative images of the staining. Positive staining is observed in all 3 experimental groups, with a higher presence in the TG+C group. Red-stained lipid droplets are observed within hepatocytes, surrounding the nucleus. B. Quantification of the percentage of stained area in the field observed for each experimental group. Data are shown as mean±SD. Statistical analysis performed using one-way ANOVA followed by Tukey's post-hoc analysis. *p<0.05, ****p<0.0001. Scale bar: 50 μm.

FIG. 14. Sudan Black B staining in liver tissue from WT and TG mice fed a C or F diet. A. Representative images of the staining. Positive staining is observed in all 3 experimental groups, with WT+C and TG+C mice showing a greater amount of lipofuscin, stained blue and distributed within the hepatocytes surrounding the cell nucleus. B. Quantification of the percentage of stained area in the field observed for each experimental group. Data are shown as mean±SD. Statistical analysis performed using one-way ANOVA followed by Tukey's post-hoc analysis. *p<0.05. Scale bar: 50 μm.

FIG. 15. Representative images of hematoxylin and eosin staining. Evidence of lipid accumulation in the form of macro- and microvesicles is observed in TG+C mice. WT+C mice show minimal structural alterations. TG+F mouse tissue showed no apparent alterations. Scale bar: 50 μm.

FIG. 16. Representative images of Masson's trichrome staining. No fibrosis is observed in the area adjacent to the central vein in any of the three groups fed a control diet or a diet with soluble fiber. Scale bar: 50 μm.

FIG. 17. Presence of ceramides in astrocytes. Ir+ceramides are observed in the extension of astrocyte dendrites in the entorhinal cortex of a TG mouse. Scale bar: 50 μm.

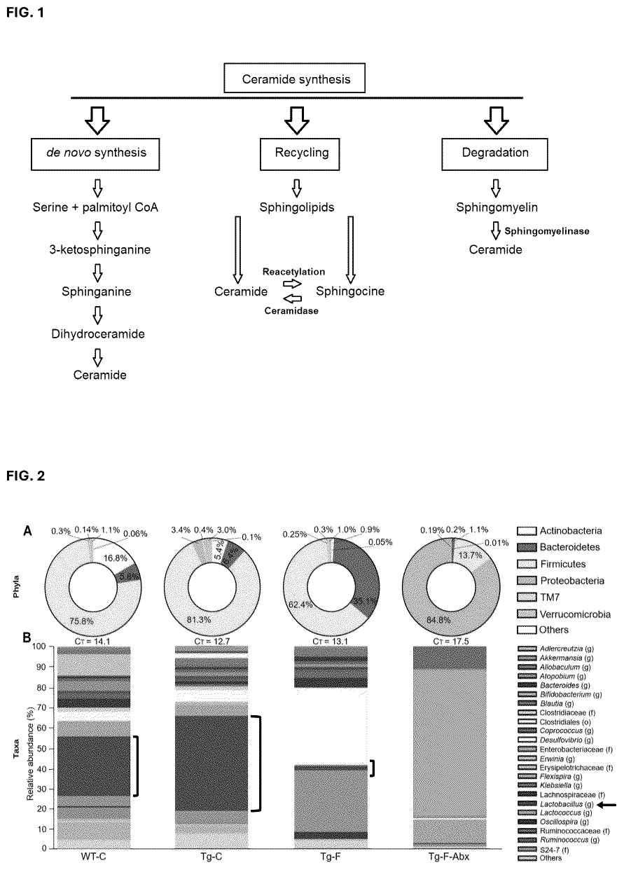

### DETAILED DESCRIPTION OF THE INVENTION

The present invention provides the use of soluble fiber to prevent lipid accumulation in the liver associated with increased ceramide levels in the brain, as seen in the murine model of Alzheimer's disease.

The experimental basis of the present invention lies in:
The determination of the presence of lipids in the liver and ceramide levels in the brain of transgenic (TG) mice, i.e., with AD, and control (healthy) mice.
The evaluation of the effect of fiber intake on lipid alterations in the liver.
To measure this lipid accumulation, liver samples were taken and stained with Oil Red O, both in TG mice and control mice.
The presence of ceramides in the brain was also identified through immunofluorescence in samples from transgenic mice and control mice.
It was also determined whether fiber intake prevents lipid accumulation in the liver of transgenic mice.

The compositions of the invention comprise fructans isolated from Agave called agavins (or soluble fiber as also referred to herein), which provide the therapeutic effects described above when consumed. For the purposes of the invention, any agavine that provides these effects may be used, such as agavines obtained from Agave tequilana Jal., Agave tequilana Gto., Agave angustifolia Oax., Agave angustifolia Son., Agave potatorum Oax., Agave cantala Oax., Agave fourcroydes Yuc., Dasylirion spp. Chih. or mixtures thereof. However, in a preferred mode of the invention, agavins obtained from Agave tequilana Jal. are most preferred.

The compositions described herein comprise Agave fructans or agavins of formula I:

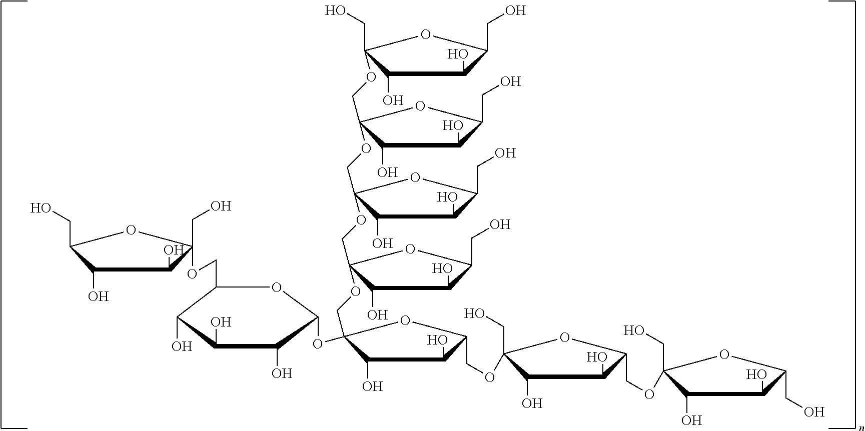

wherein n has a value of 2 to 32, which can be obtained by known methods and having the properties that have been previously described by López et al. (López, Mercedes G., et. al. 2003); likewise, said Agave fructans can be obtained from different types of Agaves. In one of their preferred forms, the compositions of the invention comprise Agave fructans, where said fructans are mixtures of Agave fructan molecules of different degrees of polymerization (DP) in various proportions, where n has a value of 2 to 32 (DP of 2 to 32) and can be obtained preferably from A. tequilana Weber var. Azul, such as a mixture containing Agave fructan molecules where n has a value of 3 to 29 (DP of 3 to 29) or a mixture containing Agave fructan molecules where n has a value of 4 to 23 (DP of 4 to 23). For the purposes of the invention, any of the mixtures of Agave fructans with a value of n from 2 to 32 (SD from 2 to 32) is useful for observing the beneficial effects described in the present invention.

In an embodiment, the agavins included in the compositions of the invention comprise the compounds of the three main groups of Agave fructans, such as groups I, II, and III, classified based on the degree of polymerization (DP) and the abundance of bonds (Mancilla-Margalli and Lopez, 2006). Although the inclusion of any of these compounds or mixtures thereof in the compositions of the invention provides the beneficial effects exhibited by said compositions, the use of compounds of formula I is preferred.

The Agave fructans comprising the compositions of the present invention can be obtained by known methods (López, Mercedes G., et. al. 2003; Urias Silvas, J, et. al. 2006, López Pérez, Mercedes G; et. al. 2010, López Pérez, Mercedes G; et. al. 2012, López Perez, Mercedes G; et. al. 2013), as well as by any other method that allows said Agave fructans with the aforementioned characteristics to be obtained.

The compositions of the present invention may be administered alone or in combination with food in order to observe their therapeutic effects.

Due to the multiple beneficial effects that consumption of the compositions of the invention provides for the restoration of health, said compositions can be used to restore health parameters that have been affected by the onset of intestinal dysbiosis, for example those affected by an increase in lipids in the liver or an increase in ceramides in the brain generated by said dysbiosis, such as those that occur in Alzheimer's disease, cognitive and learning degeneration, neuroinflammation as such, as well as liver disorders such as fatty liver or liver disorders caused by an increase in lipids in patients that have been caused by said intestinal dysbiosis.

Additionally, the compositions of the invention provide very convenient prebiotic effects because their consumption stimulates the growth of probiotic bacteria better than other fructans, as they are capable of stimulating, for example, the growth of such bacteria (Coprococcus, Dehalobacterium, and Moryella, which are butyrate-producing bacterial genera, for example) due, possibly, to the branched structures of Agave fructans. The compositions of the present invention have great potential as food ingredients and/or ingredients that promote well-being and health. As will be seen below, the administration of the compositions of the invention causes substantial modifications in the intestinal microflora, generating the appearance of bacterial strains that have beneficial therapeutic effects in the treatment of conditions caused by an increase in lipids, and ceramides caused by intestinal dysbiosis, while simultaneously eliminating bacterial strains that are harmful to the health of these patients. In this regard, the present invention demonstrates that the intestinal microflora population of patients with neurodegenerative diseases such as Alzheimer's disease causes the symptoms and progression of the disease by generating substances that cause neuroinflammation, with the consequent appearance and progression of the typical symptoms of the disease, a situation that is substantially modified by the administration of the compositions of the invention, which modify the intestinal microflora by stimulating the appearance of beneficial bacterial strains that generate substances with anti-neuroinflammatory effects, such as ceramides, thus restoring the patients' normal health parameters, eliminating bacterial strains that generate factors that cause neuroinflammation, and consequently eliminating the neuroinflammation present in the patient.

The compositions of the invention may also include components that do not inhibit the prebiotic effect and the reduction in food and energy intake and that at the same time allow for various presentations or improvements in the taste or appearance of the compositions, such as excipients, flavorings, stabilizers, preservatives, or adjuvants commonly known in food preparation.

For the purposes of the invention, the compositions described herein comprise a concentration of Agave fructans or agavins of 3% to 20% by weight relative to the total weight of the composition, however, those with a concentration of 3% to 15% are preferred, and more preferably those with a concentration of 3% to 10%. These compositions can be administered directly or in conjunction with food and beverages, although for the purposes of the invention, it is preferred that they be administered directly and at a frequency that allows the disappearance of the symptoms of the disease to be observed, for example, by means of at least one daily dose, or 2 to 3 times per day for as long as the treating physician considers appropriate for the treatment.

It should be noted that Agave fructans are well tolerated at doses of 5 to 7.5 g/day for periods of up to 21 days, followed by a week's break (Holscher, H., et. al., 2015). Agave fructans have good water solubility, so a dosage of 3 to 20 g per day, particularly 3 to 10 g per day and more particularly 3 to 7.5 g per day (sachets to be dissolved in half a glass of water) could prevent neuroinflammation or the increase in lipids associated with intestinal dysbiosis, present in various conditions as described here.

The compositions described here can be administered in the form of solid edible compositions, powders, liquids, gels, or any other form that is convenient for consumption.

The compositions of the invention can be obtained by conventional methods known in the state of the art, by mixing the various ingredients of interest together with the selected agavins in the appropriate amounts and proportions described above. However, methods that preserve the properties of the agavins in these compositions are preferred. In one of its forms, agavins may be in the form of an extract or powder and be mixed with the rest of the selected components to achieve the w/w concentration thereof in the compositions of the invention as mentioned above.

The compositions of the invention provide greater and improved effects with a low amount of active ingredients, as well as greater beneficial effects with their consumption, such as providing prebiotic effects while providing evident and sustained therapeutic effects for the treatment of diseases caused by neuroinflammation due to an increase in ceramides, increased lipids, and intestinal dysbiosis, such as Alzheimer's disease and fatty liver disease. Because, the compositions of the invention provide greater and better benefits than compositions made up of components other than Agave fructans.

For the purposes of the present invention, transgenic mice with Alzheimer's disease (AD), (TG, APP/PS1) obtained from Jackson Laboratories #005864 expressing a humanized APP chimera (Mo/HuAPP695) and a human PS1 mutant (PS1-dE9) were obtained from the authorized supplier and bred in the CICUAL animal facility at CINVESTAV-IPN. Animal handling was established in accordance with NOM-062-ZOO-1999 and the recommendations of the CICUAL of CINVESTAV-IPN (Protocol No. 235-16). All animals were genotyped by polymerase chain reaction (PCR) using 100 ng of DNA isolated from the tails of the mice and primers for APP, PS1, and IL-2 as positive controls. Mice showing both mutations were classified as TG, and wild-type (WT) siblings were used as a control group.

This APP/PS1 mouse model is associated with familial AD, as they exhibit neuropathologies similar to the disease (accumulation of Aβ in the brain) from week 6 of age, and cognitive impairment is observed from the fourth month of life (Trinchese et al., 2004). Dysbiosis has also been reported in the intestinal microbiota of APP/PS1 mice from 8 weeks of age (Wang et al., 2020).

The invention was developed in two phases:

Phase I) Six-month-old male WT and TG mice were used, fed a standard chow diet ad libitum (WT+Chow, n=6; TG+Chow, n=6), housed under 12:12 hours of light/dark conditions.

In this phase, the relative abundance of taxa was obtained from fecal samples from these mice (WT+C, TG+C), and the integrity of the liver tissue was also evaluated using Oil Red O staining to observe lipid accumulation in the tissue.

Phase II) Male WT and TG mice were used, but now 4 months old and fed for 2 months with a control diet (AlN-93, C) (Reeves, 1997) or a control diet with soluble fiber (5% Agave fructans, F) (Bustar Alimentos, S.A. de C.V.), generating the following experimental groups:

$$WT + C = Wild - type mice on a control diet ( n = 5 ) \qquad A )$$

$$TG + C = APP / PS 1 mice on a control diet ( n = 5 ) \qquad B )$$

$$TG + F = APP / PS 1 mice on a high - fiber diet ( n = 5 ) \qquad C )$$

Also TG+F mice treated with antibiotics (TG+F+Abx).

The control diet (C) for feeding the mice was prepared with a composition of corn starch (450.1 g/kg), casein (140 g/kg), maltodextrin (169.4 g/kg), sucrose (100 g/kg), soybean oil (40 g/kg), cellulose (50 g/kg), mineral mixture (35 g/kg), vitamin mixture (10 g/kg), I-cysteine (3 g/kg), choline (2.5 g/kg), and tert-butylhydroquinone (TBHQ; 0.01 g/kg). Energy: 3.90 kcal/g.

The diet with 5% fructans (soluble fiber) (F) for feeding the mice was prepared with a composition of corn starch (400.1 g/kg), casein (140 g/kg), maltodextrin (169.4 g/kg), sucrose (100 g/kg), soybean oil (40 g/kg), cellulose (50 g/kg), mineral mixture (35 g/kg), vitamin mixture (10 g/kg), L-cysteine (3 g/kg), choline (2.5 g/kg), tert-butylhydroquinone (TBHQ; 0.01 g/kg), and fructans from Agave tequilana (50 g/kg). Energy: 3.90 kcal/g.

All animals were euthanized at 6 months of age with a dose of pentobarbital (0.5 ml, intraperitoneal). The brain was immediately removed; the two hemispheres were separated to obtain the cortex and hippocampus. The tissues were stored in 4% paraformaldehyde (4% PFA) at 4° C. or deep-frozen (−70° C.) for further study.

In Phase I of the invention, the liver was recovered and divided into three parts, one of which was stored in 4% PFA (see example H), another part was stored in ultra-freezing (−70° C.), and the remaining part, intended for the Oil Red O technique, was embedded in 2-methylbutane (Sigma-Aldrich) at liquid nitrogen temperature and then covered with a cryotomy medium (Tissue-Tek) mounted on a 2×2 cm cork panel, after which it was stored at ultra-low temperatures until use.

We evaluated the liver tissue integrity using several techniques. Oil red staining was used to observe lipid accumulation in the tissue (Mehlem et al., 2013). Oil red staining was applied in phase I.

For Phase II of the invention, the liver was recovered and divided into two parts, one stored in 4% PFA, and the other stored in ultra-freezing (−70° C.). Hematoxylin and eosin and Masson's trichrome stains were only used in phase II; Oil Red O staining was also applied in phase II. Examples A-Y describe the procedure for each of these techniques.

The hematoxylin and eosin technique was used to observe the macroscopic architecture of the tissue (Brown & Kleiner, 2016). The Masson's trichrome technique was used to evaluate fibrosis (Brown & Kleiner, 2016). Sudan Black B staining allowed lipofuscin aggregates to be observed (Evangelou & Gorgoulis, 2017).

The sections processed for Oil Red O and Sudan Black B staining were observed under an optical microscope (Leica DM500) using a 40× objective. The images were captured with a Leica ICC50 W camera, and three fields per frozen liver section per animal were obtained.

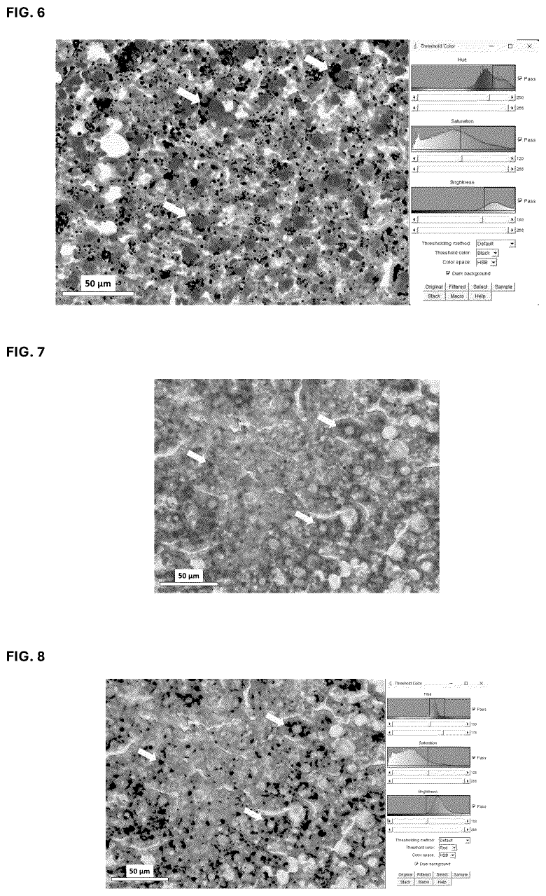

For the analysis of Oil Red O staining images, specific dye marks within the cell were identified, whose main characteristics are reddish coloration and the shape of lipid deposits, which is round-, similar to a drop (FIG. 5). Once the specific mark was identified, the photographs were analyzed in the ImageJ program in order to select and quantify the percentage of area in the photograph occupied by the dye mark (FIG. 6A).

The quantification of the Oil Red O stain was performed by adjusting the color threshold, which includes the hue, saturation, and brightness values of the image. Adjusting the hue value allowed us to select the area corresponding to the red stain, discriminating those with a different coloration, such as those belonging to the nuclei, resulting from counterstaining with hematoxylin. The saturation and brightness were adjusted in order to reduce the quantification of the dye stain outside the cell, since in this case it gives the stain a faint reddish coloration, different from the specific stain inside the cell (FIG. 6B).

As for Sudan Black B staining, specific intracellular dye marks were identified, whose main characteristic is their blue coloration (FIG. 7). The photographs were analyzed in the ImageJ program to select and quantify the percentage of area in the photograph occupied by the dye stain (FIG. 8A). The Sudan B stain was quantified by adjusting the color threshold (FIG. 8B), following the same principle as in the images for Oil Red O.

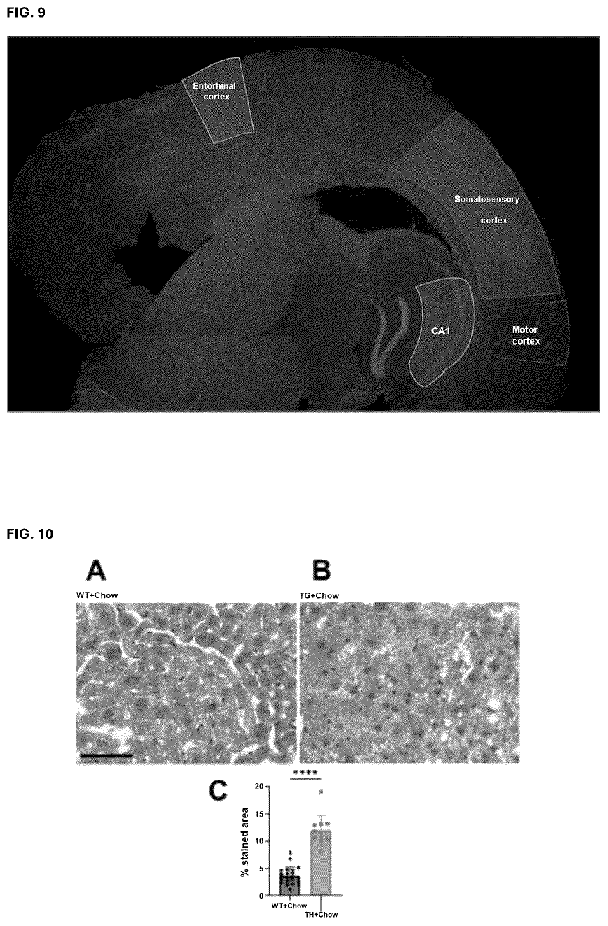

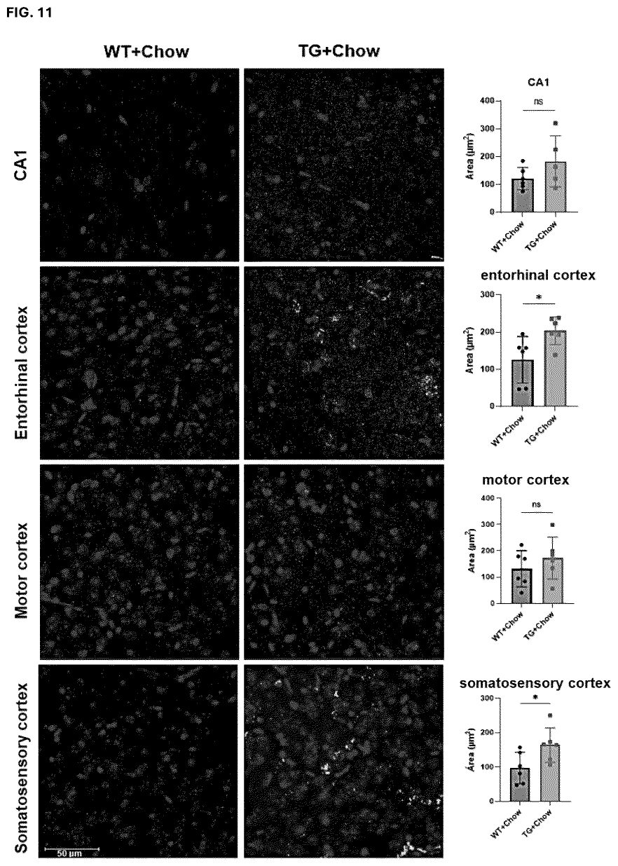

For the quantitative analysis of ceramide levels, the area occupied by the signal from the secondary antibody stain in the images captured by the Leica TCS-SP8 confocal microscope and processed in Leica LASX software was quantified. The areas of the brain photographed and processed were CA1, the primary motor cortex, the somatosensory cortex, and the entorhinal cortex (FIG. 9).

The images analyzed show the cell nuclei in blue and the immunoreactivity of ceramides in green, correspond to two fields per section, captured at 63×. Two 40 μm thick sections were analyzed and two fields per brain area were captured. The results of the quantitative analysis are the average of the fields captured.

### APP/PS1 Transgenic Mice (TG for EA) Show Greater Lipid Accumulation in the Liver Compared to WT Mice.

Oily red staining, applied to liver samples from TG+Chow and WT+Chow mice, evaluated the percentage of neutral lipids in the total image area. The presence of the specific staining marker was observed, as it is characterized by its red color and drop shape within mouse hepatocytes (FIG. 10A). In the TG+Chow group, a greater amount of specific staining was observed, with the presence of lipid microvesicles within the hepatocytes compared to WT+Chow mice (FIG. 10B). Semi-quantitative analysis confirmed that TG+Chow mice show a significantly higher accumulation of lipids compared to WT+Chow mice (p<0.0001) (FIG. 10C). These results suggest that TG mice for EA have alterations related to lipid metabolism, specifically triglycerides, at the hepatic level.

Although most studies on lipid accumulation in TG and WT mice are conducted under protocols of diet-induced obesity (high-fat diet), it has been reported that WT mice show larger lipid deposits (macrovesicles) in the liver compared to TG mice (microvesicles) (Kim et al., 2016). In another study using 6- and 10-month-old APP/PS1 mice, it was observed that, after a high-fat diet, TG mice accumulate more lipids in the liver compared to WT mice (Mengr et al., 2023).

The main form of lipid accumulation in liver of TG mice for AD was in the form of microvesicles. In NAFLD, lipid accumulation in liver can exist in two forms: macrovesicular and microvesicular. The macrovesicular form is the most common, characterized by the presence of a single lipid droplet within the cytoplasm with the ability to displace the nucleus to the cell periphery (Brunt, 2012). On the other hand, the microvesicular form is described as small droplets (less than 1 μm in diameter) around the nucleus, giving the cytoplasm a blurred appearance (Tandra et al., 2011). It has been observed that, compared to the macrovesicular form, the microvesicular form is related to mitochondrial alterations and inflammation, indicating an advanced stage of liver injury (Celebi et al., 2020; Tandra et al., 2011).

This could indicate that in TG mice there are processes in the liver that are related to microvesicular lipid accumulation, such as mitochondrial dysfunction (Celebi et al., 2020), which could also explain the alterations in fatty acid beta oxidation that induce the generation of lipotoxic species such as ceramides and increased lipid accumulation (Hensley et al., 2000; Koo, 2013).

The data presented corroborate and support previous results and suggest that lipid accumulation in the liver is an early event that occurs spontaneously in the APP/PS1 mouse model.

Ceramides have the ability to be released into the circulation and transported to different organs, including the brain. Based on this information, the presence of ceramides in the brains of the study mice was analyzed by immunofluorescence.

### Ceramide Levels are Increased in the Brains of APP/PS1 Transgenic Mice.

The presence of ceramides in the brains of TG and WT mice was determined using the immunofluorescence technique. In the CA1 region of the hippocampus, positive immunoreactivity of ceramides (ceramides+ir) was observed in both WT+Chow and TG+Chow mice. In TG+Chow mice, the presence of ceramides was localized in the brain parenchyma and in perinuclear cells with a dotted pattern (FIG. 11A, B).

Quantitative analysis of the area occupied by ceramides+ir in CA1 showed no significant difference between the two experimental groups (p=0.1691) (FIG. 11I). In the entorhinal cortex, TG+Chow mice showed greater aggregation of ceramides in the perinuclear space compared to samples from the WT+Chow group (FIG. 11C, D). Analysis of the area occupied by the signal showed significant differences between the experimental groups in the entorhinal cortex (p=0.0255) (FIG. 11J). In the motor cortex, ceramides were present in both experimental groups (FIG. 11E, F), with no significant differences between them (p=0.3685) (FIG. 11K).

In the somatosensory cortex, a dotted pattern of ceramides+ir was observed in the tissue of both experimental groups, although with greater intensity in the TG+Chow group (FIG. 11G, H).

Quantitative analysis showed a significant difference between both experimental groups in the somatosensory cortex (p=0.0374) (FIG. 11L).

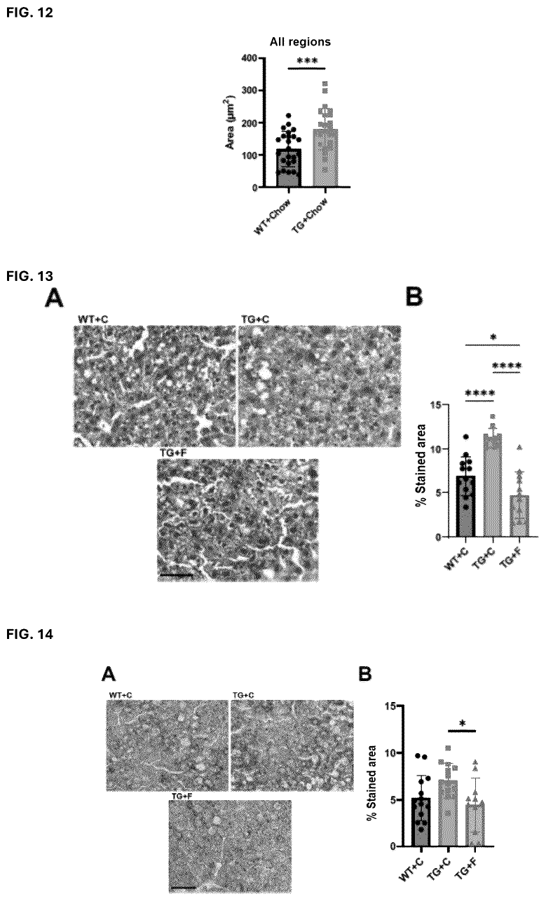

Quantitative analysis was performed on all images acquired from all brain regions, and significant differences were observed between the groups (p=0.0008) (FIG. 12).

The data obtained indicate an increase in ceramides in the brains of TG and mice, related to the levels of lipids accumulated in the liver.

Differences in lipid metabolism between TG and WT mice have been attributed to genotype differences (Bai et al., 2020), but this conclusion has not been extensively explored. It has recently been proposed that in APP/PS1 mice and mice treated with intracerebroventricularly injected Aβ42 peptide, levels of phosphorylated tau and ceramides are increased in the circulation, causing endoplasmic reticulum stress in the hypothalamus, affecting the expression of genes related to lipid metabolism in the liver and consequently increasing the synthesis of free fatty acids and ceramides (Liu et al., 2024). It has been shown that in patients with AD and other neurodegenerative diseases, such as PD, there is an accumulation of ceramides in the brain (Wang & Bieberich, 2018).

The presence of ceramides in AD has been linked to various neuropathological markers, such as the generation of Aβ peptide, oxidative stress, and neuronal death (Jana et al., 2009). Furthermore, it has been reported that in the brains of TG mice and in the brains of patients with AD (post-mortem), there are alterations in ceramide synthesis via sphingomyelin degradation; in addition, alterations in various genes related to ceramide synthesis have been reported in these patients (Baloni et al., 2022).

In addition, the immunoreactivity against ceramides observed in this study corresponded to that of the Cer16 and Cer24 species, according to the antibody used (Enzo Life Science Cat. No. ALX-804-196). It has been reported that in brain samples from patients with AD, the species Cer16, Cer18, Cer20, and Cer24 are higher compared to samples from healthy subjects; in addition, it has been observed that the levels of these species increase in patients with more than one neuropathological abnormality (Filippov et al., 2012).

In another transgenic mouse model for AD, with a mutation in PS1, it was reported that ceramides C20 and C24 accumulate 4 and 8.5 times more, respectively, in the brain compared to WT mice (Wang et al., 2008).

With regard to glial cells in the brain, it has been reported that astrocytes are sensitive to ceramide-mediated apoptosis, specifically by Cer20 (Wang et al., 2008).

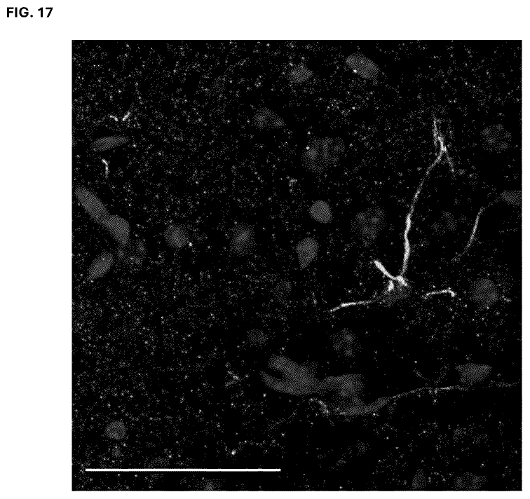

It should be noted that in our results, the immunofluorescence signal in the brain samples from TG mice resembles glial processes (FIG. 17), specifically astrocytes. Astrocytes are a type of glial cell present in all regions of the brain, whose functions range from forming the blood-brain barrier and providing metabolic support to neurons to repairing and protecting brain tissue (Ransom & Ransom, 2012). In AD, astrocytes are overactivated, significantly modifying their physiological functions to a pathological state where there is an increase in the recruitment of immune cells to brain tissue, dysfunction of the blood-brain barrier, and alterations in synapses (Cabezas et al., 2014; Verkhratsky et al., 2010). It has been detailed that astrocytes participate in the degradation of the Aβ peptide and, paradoxically, also in its production (Verkhratsky et al., 2010). Because it is important to identify astrocytes in the pathogenesis of AD, as this could explain in detail the relationship between their presence and the environment in which they are found. Furthermore, in the brains of patients with AD, there is abnormal accumulation of ceramides in astrocytes in the cerebral cortex (Satoi et al., 2005). and in a cell model of neuroblastoma, it was reported that ceramide levels increase extracellularly and intracellularly as a result of retinoic acid-induced neuronal apoptosis (Satoi et al., 2005). This could indicate that astrocytes are capable of generating and releasing ceramides extracellularly to initiate apoptotic processes in neurons. In order to corroborate the presence of ceramides in the astrocytes of TG mice, double immunofluorescence (ceramide+astrocytes) is necessary to determine the specific cell type.

Regarding the regional distribution of ceramides in the brain, it was observed that in TG mice there was a greater accumulation in the entorhinal and somatosensory cortex regions compared to WT mice. The presence of ceramides in these regions is relevant, as it is recognized that vulnerable regions of the brain in AD include the cortex and hippocampus, specifically the entorhinal and CA1 regions, respectively (Wang et al., 2010), as it is known that neurons belonging to these regions show large accumulations of neurofibrillary tangles that promote cell death in the early stages of AD (Price et al., 2001).

Although the somatosensory cortical region is not described as a particularly vulnerable region in AD, it has been reported that, in a rat model for AD, there is an arterial accumulation of the Aβ peptide starting in the somatosensory cortex (Bishay et al., 2022). This result is very important, as cerebral amyloid angiopathy has been observed to be a pathology associated with AD, with a prevalence in AD patients of 80-90% (Greenberg et al., 2020). In our study, we observed that ceramide accumulation in the CA1 region was slightly higher in TG mice than in WT mice, but no significant differences were found between groups. With regard to the motor cortex, no significant differences were observed between groups, as it has been reported that the neuropathological changes of AD only appear in the motor cortex in the late stages of the disease (Ferreri et al., 2003).

According to various studies, the synthesis and accumulation of ceramides in the brains of AD patients is mediated by alterations in sphingolipid metabolism and by various enzymes involved in de novo synthesis and synthesis by sphingomyelin degradation, through the upregulation of ceramide synthases and sphingomyelinases, respectively (Baloni et al., 2022; Filippov et al., 2012). In addition to the evident local production of ceramides in the brain, it has been proposed that the accumulation of ceramides in the brain could also have a systemic origin. This has been demonstrated in a study where it was observed that intraperitoneal administration of a ceramide analog, Cer2, for 7 alternate days in rats from 3 to 30 days of age, can induce cognitive and motor alterations through changes in insulin signaling and the activation of proinflammatory cytokines (De La Monte et al., 2010) It is suggested that ceramides, being lipid in nature, are capable of crossing the blood-brain barrier and generating the proposed changes in the brain. This work emulates the conditions of various systemic disorders, such as obesity, type 2 diabetes, or NAFLD, where there is an exacerbated production of ceramides, so the mechanism described could explain the relationship between these diseases and neurodegeneration (Craft, 2007; De La Monte & Tong, 2014; Tong & De La Monte, 2009).

The results of the present invention, with 6-month-old TG mice in the early/moderate stage of the disease, indicate that ceramides are present in the brain from the early stages of the disease, serving as a possible biomarker of the pathology. In order to determine with certainty, the origin of ceramides in the brain and the possible signaling between the liver and the brain, it is necessary to determine levels of ceramide in the liver, plasma, and brain of the study groups, which will give us more information about the interaction between these organs.

It is known that a high-fiber diet can improve the lipid profile in obese patients and that in TG mice with AD it can modify the intestinal microbiota. Thus, we evaluated whether soluble fiber consumption could prevent accumulation and alter s in lipid metabolism in the livers of TG mice.

### Soluble Fiber Intake Prevents Lipid Accumulation in the Liver of APP/PS1 Transgenic Mice.

We evaluated whether lipid accumulation in the liver can be prevented by consuming a diet rich in soluble fiber (fructans). After two months of intervention, TG+C mice showed greater lipid accumulation compared to WT+C and TG+F mice, with abundant red staining in the form of small droplets (microvesicles) inside the hepatocytes of the TG+C group (FIG. 13A). In the TG+F group, a reduction in lipid accumulation was observed (FIG. 13A). Semi-quantitative analysis confirmed these observations, showing significant differences between TG+C mice and all experimental groups (TG+C vs. WT+C, p<0.0001; TG+C vs. TG+F, p<0.0001); fiber intake for two months helped prevent lipid accumulation even at levels significantly lower than those in the WT+C group (p=0.0387) (FIG. 13B).

Because lipid accumulation was observed in TG+C mice, we evaluated whether other processes related to lipid metabolism, such as lipofuscin production and accumulation, could be modulated by soluble fiber intake.

### Soluble Fiber Intake Prevents Lipofuscin Accumulation in the Liver of Transgenic Mice.

Lipofuscin accumulation was determined using Sudan Black B staining. Semi-quantitative analysis of histological sections of liver tissue allowed us to determine the effect of soluble fiber intake on lipofuscin accumulation. A greater amount of specific blue staining was observed in TG+C mice compared to the other groups.

This staining could be seen surrounding the nucleus of hepatocytes throughout the entire field of view (FIG. 14A).

Significant differences were observed between the TG+C and TG+F groups (p=0.0285) (FIG. 14B), where the data obtained suggest that lipofuscin accumulation is possibly modulated by soluble fiber intake. Soluble fiber intake is capable of modulating lipid accumulation in the liver and possibly inducing better overall liver function. Based on this argument, the morphological state of the liver tissue was analyzed through histological staining.

### Soluble Fiber Intake Improves Liver Tissue Integrity.

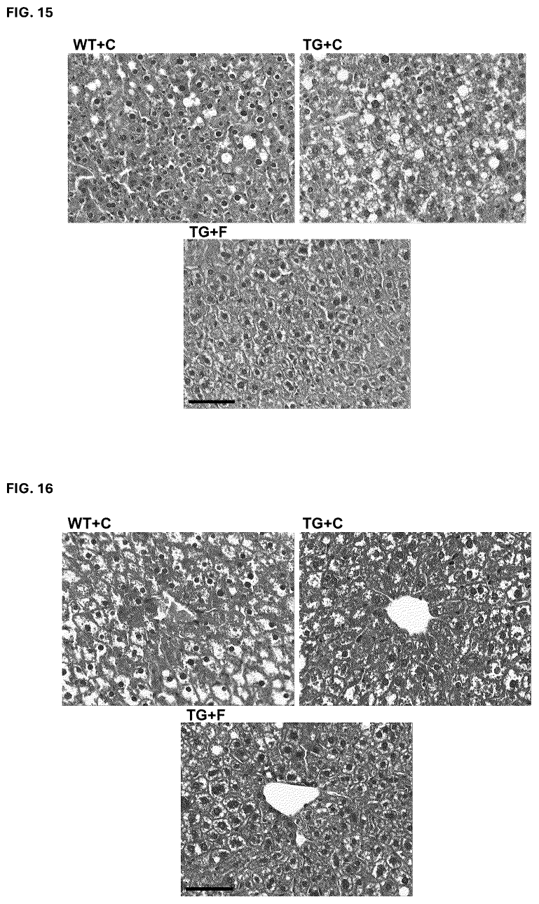

Hematoxylin and eosin staining was used to assess the overall macroscopic condition of the tissue (FIG. 15). After evaluating the samples, various structural and cellular alterations in the liver tissue were revealed in the TG+C group of mice. The presence of macro- and microvesicles of lipids was observed in much of the tissue in the mice. In contrast, the WT+C mice had more uniform tissue, but with some signs of lipid accumulation. On the other hand, the TG+F mouse sections showed uniform tissue with visible cytoplasm, nucleus, and cell membrane, without any apparent alterations such as those observed in the TG mice without fiber.

These results suggest that fiber could improve the overall condition of the liver in transgenic mice by preventing lipid accumulation.

Based on this, it was decided to analyze whether fiber intake is capable of reversing more advanced stages of NAFLD, such as fibrosis.

Masson's trichrome staining, aimed at visualizing type 1 collagen fibers stained blue, revealed no fibrosis in any of the four experimental groups. FIG. 16 shows representative images of the central veins, where there are clearly no extensions of collagen fibers into the liver parenchyma. These results indicate that the lipid accumulation observed with Oil Red O and hematoxylin and eosin staining is at a stage where liver damage is not severe enough to cause fibrosis in the tissue.

Soluble fiber intake has recently been considered as a potential treatment for various metabolic disorders such as obesity, insulin resistance, and NAFLD, through the stimulation of satiety, reducing the number of times the individual needs to eat and thus inducing caloric restriction and weight loss (de Oca et al., 2020; Parnell et al., 2012; Solah et al., 2017).

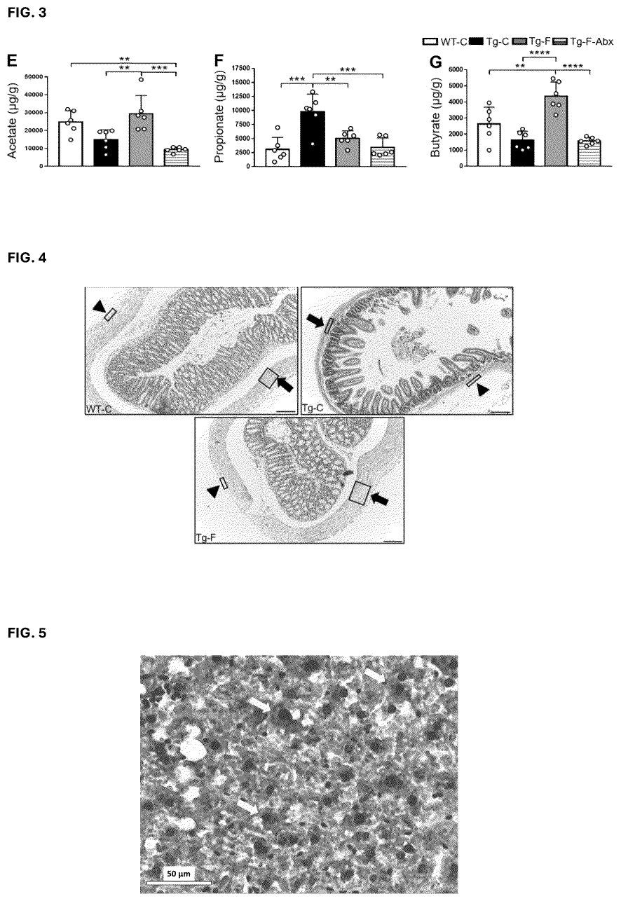

We have data showing that TG+C mice had intestinal dysbiosis, which was reduced after fructan intake. Among the most abundant taxa in TG mice was Lactobacillus, a taxon that has been associated with ceramide accumulation in the liver (Jiang et al., 2015; Wang et al., 2019); while the TG group treated with fructans showed a significant decrease in this taxon. In a previous study conducted by our research group using TG, APP/PS1 mice fed with fructans from Agave tequilana, cognitive improvement, reduced anxiety, and reduced neuroinflammation were observed, all of which are markers related to the restoration of the intestinal microbiota observed (FIG. 2) and the production of short-chain fatty acids (FIG. 3). Short-chain fatty acids, such as butyrate, are capable of improving cell function by serving as energy substrates (Zhang et al., 2021) or reducing inflammation (Segain, 2000). In this same study, a restoration of intestinal morphology was observed, as damage was observed in TG mice, which was reversed by fiber intake (FIG. 4).

It has been proposed that NAFLD may be related to the abundance of certain bacterial populations, specifically Lactobacillus and Bacteroides, and therefore to the production of BSH in the intestine (Wang et al., 2019). This could be related to our data, where TG+F mice, which showed a decrease in Lactobacillus, also showed low levels of lipids in the liver.

Furthermore, TG+F mice show an increase in the abundance of butyrate-producing bacteria (FIG. 3), a short-chain fatty acid with beneficial effects for the host. Probiotic treatment has been shown to promote butyrate production, attenuating the progression of NAFLD-associated pathologies, such as lipid accumulation in the liver, steatohepatitis, and hepatocarcinogenesis, through improved lipid metabolism and reduced oxidative stress in the livers of rats (Endo et al., 2013). Similarly, it has been observed that, in mice fed a high-fat diet, treatment with butyrate is capable of reducing lipid accumulation in the liver and upregulating the GLP-1 receptor (glucagon-like peptide-1) receptor, which is related to metabolic processes and whose expression is attenuated in the liver of patients with NAFLD (Zhou et al., 2018). Another important effect of butyrate on liver function is through the restoration of intestinal permeability (Amiri et al., 2022). TG+F mice showed an improvement in intestinal morphology compared to TG+C mice, which had damage to the intestinal epithelium (FIG. 4), which is consistent with several reports in animal and cellular models indicating that butyrate treatment is capable of maintaining intestinal homeostasis through the regulation of various proteins related to tight junctions in the tissue (Jin et al., 2016; Ma et al., 2012).

The presence of lipofuscin, an aggregate of oxidized and cross-linked proteins, lipids, and metals that cannot be degraded by cells, has been attributed to senescence, the response of cells to oxidative stress, and the alteration of lipophagy, a type of autophagy specific to lipids, in NAFLD (Gao et al., 2020; Saif et al., 2020). A high-fat diet has been shown to induce lipofuscin accumulation in the liver of rats, in addition to an increase in other markers related to liver senescence, such as p16 and p27, proteins related to cell cycle arrest (Gao et al., 2020). Lipofuscin accumulation has also been observed in a model of mice treated with carbon tetrachloride, a model of liver damage induction by reactive oxygen species (Saif et al., 2020).

The present invention shows that fiber intake is capable of preventing lipofuscin accumulation in the liver in TG mice, compared to mice fed a control diet. This effect could be attributed to changes in the intestinal microbiota and increased butyrate production, as butyrate has been shown to reduce oxidative stress caused by a high-fat diet in rats (Sun et al., 2019).

Several animal studies have observed that soluble fiber intake can reduce triglyceride accumulation in the liver (Daubioul et al., 2000; Sugatani et al., 2006; Wada et al., 2005). The effects of soluble fiber on NAFLD have been attributed to various mechanisms, such as weight loss, reduction of adipose tissue, improvement of glucose metabolism, and modification of the gut microbiota (Parnell et al., 2012). Furthermore, fiber intake also restored liver morphology, as assessed by hematoxylin and eosin histological staining. The histological characteristics of liver damage in TG+C mice are consistent with those described in the scientific literature (Brown & Kleiner, 2016). However, no fibrosis was observed in any of the three experimental groups, which could indicate that liver damage is in its very early stages, where the synthesis of collagen and other components of the extracellular matrix by activated hepatic stellate cells is not yet present (Khanam et al., 2021; Rinella & Sanyal, 2016).

We can therefore conclude that TG mice fed fructans prevent lipid accumulation in the liver and alterations in cell morphology, an effect mediated by changes in the intestinal microbiota and butyrate production. It would be very relevant to evaluate the effect of various prebiotics and probiotics on the modulation of other bacterial taxa and the production of short-chain fatty acids to better delineate the mechanism of action of fructans on the gut-liver microbiota axis. In this way, Agave fructans can be positioned as an effective treatment to prevent systemic events in the early stages of AD.

In present invention, an accumulation of lipids in the liver and ceramides in the brain of 6-month-old male APP/PS1 mice was observed. The presence of lipid microvesicles in the hepatic parenchyma of TG mice was accompanied by an increase in ceramides in the brains of the mice, in regions altered in AD patients. This allows us to hypothesize that there is an alteration in the liver-brain axis in TG mice. These data highlight the importance of the animal model used in the present invention, as the pathological conditions observed resemble those reported in patients suffering from AD and/or NAFLD or other related metabolic conditions.

The present invention provides the possibility of treating or preventing AD by ingesting soluble fiber for 2 months, which had a positive effect on preventing the accumulation of lipids and lipofuscin in the liver of TG mice, possibly through the restoration of intestinal dysbiosis (previously reported) and the production of metabolites such as butyrate, a short-chain fatty acid that has been attributed with multiple systemic and central benefits. This invention demonstrates the therapeutic role of soluble fiber at both the central and peripheral levels.

Although the relationship between the liver and brain needs to be satisfactorily demonstrated, our data add information to the characterization of the TG mouse model for AD, in the field of lipid alterations, which could be relevant in identifying risk factors and possible markers of AD in the early stages of the disease.

The object of the invention is to provide the basis for obtaining or preparing soluble fiber compositions, as active ingredients, to prevent the accumulation of ceramides in the liver and lipids in general in the brain, thereby preserving the integrity of liver tissue and, for the effects on the brain, also aiding in the prevention of Alzheimer's disease.

It is a modality of the invention to provide the basis for obtaining or preparing compositions of fructans, more particularly Agave fructans, as active ingredients, to prevent the accumulation of ceramides in the liver and lipids in general in the brain, thereby preserving the integrity of liver tissue and also aiding in the prevention of Alzheimer's disease.

The following examples are presented to provide techniques for validating the invention and are not limiting to it.

### Example 1: Treatment of Liver Tissue

A Portion of Mouse Liver, Frozen and stored in 4% PFA, was cut into quadrangular shapes, 5 mm per side. The resulting sections were fixed in 30% sucrose. The tissue was then processed to obtain sections 8 and 16 μm thick using a Leica CM1520 cryostat. Finally, the sections were stored at room temperature until use. For the Oil Red O technique used in Phase I, the tissue was processed in the cryostat to obtain sections 8 μm thick.

### Example 2: Oil Red Staining

Oil Red Staining, for the Marking of Neutral Lipid accumulation, was performed on 8 μm thick liver sections. First, the sections were hydrated in double-distilled water for 30 seconds. Next, 1 ml of 60% propylene glycol (J. T. Baker) was added to cover all sections for 30 seconds, immediately followed by 1 ml of Oil Red solution (Sigma-Aldrich) at 60° C. to cover all sections for 3-5 minutes. Once the time had elapsed, the sections were rinsed with 60% propylene glycol (J. T. Baker) and then with running water. Counterstaining was performed by adding 1 ml of hematoxylin solution (Sigma Aldrich) to cover all sections for 20 seconds. Finally, the sections were rinsed with running water for 1 minute and mounted in glycerin gelatin.

### Example 3: Sudan Black B Staining

Sudan Black B Staining, to Identify lipofuscin, was performed on 16 μm thick liver sections. The sections were hydrated in double-distilled water for 15 seconds, then washed three times with 1% PBS for 10 minutes. Once the washes were complete, the sections were incubated with 0.1% Sudan Black B solution (Sigma-Aldrich) for 1 minute, then rinsed with 1% PBS for 10 minutes three times; once finished, the sections were left to dry at room temperature for 24 hours. The sections were mounted with glycerin gelatin.

### Example 4: Masson's Trichrome Staining

Masson's trichrome staining was performed on 8 μm thick liver sections. The sections were deparaffinized and hydrated. They were then treated with Bouin's solution for one hour at 60° C., allowed to cool, and washed with running water until the dye was rinsed out; the final wash was with distilled water. The sections were then stained with ferric hematoxylin (Hycel) for 12 minutes, immediately followed by washing with running water to remove excess dye, with the final wash being with distilled water. Subsequently, the sections were stained with Biebrich's acid fuchsin-scarlet (Hycel) and washed with running water until the excess dye was removed, with the final wash being with distilled water. After washing, the sections were differentiated with phosphotungstic-phosphomolybdic acid solution (Hycel) for 15 minutes, after which they were rinsed with distilled water. Next, a counterstain with aniline blue (Hycel) was performed for 10 minutes, and the sections were washed with running water and rinsed with distilled water. The sections were then differentiated with 1% acetic acid for 1 minute and rinsed with distilled water. Finally, the sections were dehydrated with 96% xylene and mounted in resin.

### Example 5: Hematoxylin and Eosin Staining

Hematoxylin and eosin staining was performed on 8 μm thick liver sections. The sections were deparaffinized and hydrated. They were then stained with filtered Harris' hematoxylin (Biopack) for 15 minutes; the sections were washed with running water for 2 minutes. The sections were then differentiated with 1% acid alcohol for 1 minute and rinsed with running water for another minute. The sections were then placed in an ammoniacal water solution until they turned blue; they were then washed in running water for 10 minutes and placed in 80% ethanol for 2 minutes. Counterstaining was performed with eosin solution for 2 minutes. Finally, the sections were dehydrated in 96% xylene and mounted in resin.

### Example 6: Immunofluorescence in Brain

Immunofluorescence was performed on 40 μm thick brain sections obtained with a sliding microtome (Leica RM2235). The brain sections were washed in 1% PBS overnight at 4° C.

The sections were then incubated with sodium borohydride (NaBH4) (Aldrich No. 198072) for 10 minutes. Next, the sections were washed three times for 5 minutes with 1×PBS and permeabilized with 0.2% PBS-Triton for 20 minutes. The sections were treated with the blocking solution for 30 minutes (2% bovine serum albumin, BSA).

After the incubation period, the sections were washed three times for 10 minutes with 0.2% Triton PBS. The sections were then treated with the blocking antibody for one hour (Anti-Mouse IgM, 1:200; Jackson Immuno Research Cat. No. 115-005-020). The sections were then washed three times for 10 minutes with 0.2% Triton PBS.

The sections were then incubated with the primary antibody (Ceramide, 1:250, Enzo Life Science Cat. No. ALX-804-196) overnight at 4° C. Once incubation was complete, the sections were washed three times for 10 minutes with 0.2% Triton PBS. Subsequently, the sections were incubated with the secondary antibody (1:500, Jackson Immuno Research No. Cat. 115-545-020) for 2 hours at room temperature. At the end of the incubation period, the sections were washed three times for 10 minutes with 0.2% Triton PBS and then incubated with 4′,6-diamidino-2-phenylindole (DAPI; Invitrogen Cat. No. D1306) for 30 minutes at room temperature.

The sections were then washed three times for 10 minutes with 0.2% Triton PBS and finally mounted on slides and mounting medium (VectaShield, Vector Laboratories). For the negative control, the sections were processed according to the protocol described above, except for incubation with the primary antibody.

### Example 7: Statistical Analysis

The data presented are expressed as the mean±standard deviation (SD). The normal distribution of the data was determined using the Shapiro-Wilk test. The differences between the WT+Chow and TG+Chow groups were compared using the unpaired Student's t-test.

The differences between the experimental groups WT+C, TG+C, and TG+F were compared using one-way analysis of variance (ANOVA) followed by Tukey's post-hoc analysis. All results were considered statistically different at p<0.05.

### REFERENCES

Aburasayn, H., et al (2016). Am J Physiol Endocrinol Metab, 311, 423-435. https://doi.org/10.1152/ajpendo.00133.2016.
Amiri, P., et al (2022). Frontiers in Nutrition. https://doi.org/10.3389/fnut.2022.1037696
Angelopoulos, T. J., et al. (2009). Journal of Nutrition, 139(6). https://doi.org/10.3945/jn.108.098194
Asti, A., et. al. (2014). Journal of Alzheimer's Disease, 39(1), 169-179. https://doi.org/10.3233/JAD-131394
Auguet, T., et. al. (2014). International Journal of Molecular Sciences, 15(12), 22173-22187. https://doi.org/10.3390/ijms151222173
Bai, Y. R., et al. (2020). Neuroscience Letters, 738. https://doi.org/10.1016/j.neulet.2020.135401
Baker, L. D., et al (2011). Archives of Neurology, 68(1), 51-57. https://doi.org/10.1001/archneurol.2010.225
Baloni, P., et al (2022). Communications Biology, 5(1), 1074. https://doi.org/10.1038/s42003-022-04011-6
Bassendine, M. F., et al. (2020). Journal of Alzheimer's Disease, 75(1), 1-14. https://doi.org/10.3233/jad-190848
Bergheim, I., et al. (2008). Journal of Hepatology, 48(6), 983-992. https://doi.org/10.1016/j.jhep.2008.01.035
Bishay, J., et al. (2022). Scientific Reports, 12(1). https://dos.org/10.1038/s41598-022-19549-y
Boon, J., et. al. (2013). Diabetes, 62(2), 401-410. https://doi.org/10.2337/db12-0686
Breijyeh, Z., et al. (2020). Molecules, 25(24). https://doi.org/10.3390/MOLECULES25245789
Bril, F., et al. (2017). Hepatology, 65(4), 1132-1144. https://doi.org/10.1002/hep.28985
Brown, G. T., et. al. (2016). Metabolism: Clinical and Experimental, 65(8), 1080-1086. https://doi.org/10.1016/j.metabol.2015.11.008
Brunt, E. M. (2012). Digestive Diseases, 30 (SUPPL. 1), 61-68. https://doi.org/10.1159/000341127
Cabezas, R., et. al. (2014). Frontiers in Cellular Neuroscience, 8. https://doi.org/10.3389/fncel.2014.00211
Cai, Y., et al. (2015). Clinical Interventions in Aging, 10, 1163-1172. https://doi.org/10.2147/CIA.S85808
Castro, B. M., et. al. (2014). Progress in Lipid Research, 54(1), 53-67. https://doi.org/10.1016/j.plipres.2014.01.004
Celebi, G., et al. (2020). Acta Gastroenterol. Belg. 83. 565-570.
Chand, D., et al. (2017). Biochimica et Biophysica Acta—General Subjects, 861(1), 2981-2991. https://doi.org/10.1016/j.bbagen.2016.09.024
Chavez, J. A., al. (2012). Cell Metabolism, 15(5), 585-594. et https://doi.org/10.1016/j.cmet.2012.04.002
Chen, G. F., et. al. (2017). Acta Pharmacologica Sinica, 38(9), 1205-1235. Nature Publishing Group. https://doi.org/10.1038/aps.2017.28
Cheng, Y., et. al. (2020). Translational Neurodegeneration, 9(1). BioMed Central Ltd. https://doi.org/10.1186/s40035-020-00195-1
Colombini, M. (2010). Biochimica et Biophysica Acta-Bioenergetics, 1797(6-7), 1239-1244. https://doi.org/10.1016/j.bbabio.2010.01.021
Coppin, G., et. al. (2014). Neuropsychologia, 65, 146-155. https://doi.org/10.1016/j.neuropsychologia.2014.10.004
Cox, L. M., et. al. (2014). Cell, 158(4), 705-721. https://doi.org/10.1016/j.cell.2014.05.052
Craft, S. (2007). Current Alzheimer Research (Vol. 4).
Cutler, R. G., et. al. (2004). Proc Natl Acad Sci USA 101:2070-2075. https://doi.org/10.1073pnas.0305799101
Dabke, K., et. al. (2019). Journal of Clinical Investigation, 129(10), 4050-4057). https://doi.org/10.1172/JCI129194
Daubioul, C. A., et. al. (2000). J. Nutr, 130. https://academic.oup.com/jn/article-abstract/130/5/1314/4686540
De La Monte, S. M., et. al. (2014). Biochemical Pharmacology, 88(4), 548-559. https://doi.org/10.1016/j.bcp.2013.12.012
De La Monte, S. M., et. al. (2010). Journal of Alzheimer's Disease, 21(3), 967-984. https://doi.org/10.3233/JAD-2010-091726
De La Monte, S. M., et. al. (2005). Journal of Alzheimer's Disease, 7.
de Oca, A. P. M., et. al. (2020). Nutrients, 12(10), 1-9. https://doi.org/10.3390/nu12103100
Den Hoedt, S., et al. (2021). Frontiers in Aging Neuroscience, 13. https://doi.org/10.3389/fnagi.2021.765252
Dinkins, M. B., et. al. (2015). Journal of Alzheimer's Disease, 46(1), 55-61. https://doi.org/10.3233/JAD-150088
Dye, L., et. al. (2017). Proceedings of the Nutrition Society, 76(4), 443-454. https://doi.org/10.1017/S0029665117002014
Endo, H., et. al. (2013). PLoS ONE, 8(5). https://doi.org/10.1371/journal.pone.0063388
Espeland, M. A., et. al. (2018). Journals of Gerontology—Series A Biological Sciences and Medical Sciences, 73(4), 484-491. https://doi.org/10.1093/gerona/glx165
Esquerda-Canals, G., et. al. (2017). Journal of Alzheimer's Disease, 57(4), 1171-1183. https://doi.org/10.3233/JAD-170045
Evangelou, K., et. al. (2017). Methods in Molecular Biology, 1534, 111-119. https://doi.org/10.1007/978-1-4939-6670-7_10
Feart, C., et al. (2015). Current Opinion in Clinical Nutrition and Metabolic Care, 18(1), 51-62. https://doi.org/10.1097/MCO.0000000000000131
Ferreri, F., et. al. (2003). Ann Neurol, 53.
Field, B. C., et. al. (2020). Frontiers in Endocrinology, 11. Frontiers Media S.A. https://doi.org/10.3389/fendo.2020.569250
Filippov, V., et. al. (2012). Journal of Alzheimer's Disease, 29(3), 537-547. https://doi.org/10.3233/JAD-2011-111202
Friedman, S. L., et. al. (2018). Nature Medicine, 24(7), 908-922. https://doi.org/10.1038/s41591-018-0104-9
Galadari, S., et. al. (2013). Lipids in Health and Disease, 12(1). https://doi.org/10.1186/1476-511X-12-98
Gali, C. C., et. al. (2019). Molecular and Cellular Neuroscience, 99. https://doi.org/10.10164/j.mcn.2019.103390
Gao, Y., et. al. (2020). Redox Biology, 36. https://doi.org/10.1016/j.redox.2020.101635
Giau, V. van, et. al. (2015). Neuropsychiatric Disease and Treatment, 11, 1723-1737. https://doi.org/10.2147/NDT.S84266
Graham, L. C., et. al. (2016). Scientific Reports, 6. https://doi.org/10.1038/srep21568
Greenberg, S. M., et al. (2020). Nature Reviews Neurology, 16(1), 30-42. https://doi.org/10.1038/s41582-019-0281-2
Guan, Z. W., et. al. (2021). Molecules, 26(22). https://doi.org/10.3390/molecules26226802
Hajduch, E., et al. (2021). Journal of Clinical Medicine, 10(4), 1-19. https://doi.org/10.3390/jcm10040792
Haleem, D. J., et al. (2021). Nutritional Neuroscience, 24(3), 226-235. https://doi.org/10.1080/1028415X.2019.1619983
Harach, T., et. al. (2017). Scientific Reports, 7. https://doi.org/10.1038/srep41802
Haus, J. M., et. al. (2009). Diabetes, 58(2), 337-343. https://doi.org/10.2337/db08-1228
Hensley, K., et al. (2000). Carcinogenesis, 21(5).
Jana, A., et al. (2009). Journal of the Neurological Sciences, 278(1-2), 5-15. https://doi.org/10.1016/j.jns.2008.12.010
Jandhyala, S. M., et. al. (2015). World Journal of Gastroenterology, 21(29), 8836-8847. https://doi.org/10.3748/wjg.v21.i29.8787
Jazvinšćak Jembrek, M., et. al. (2015). Oxidative Medicine and Cellular Longevity, 2015. https://doi.org/10.1155/2015/346783
Jiang, C., et. al. (2015). Journal of Clinical Investigation, 125(1), 386-402. https://doi.org/10.1172/JCI76738
Jiang, C., et. al. (2015). Nature Communications, 6. https://doi.org/10.1038/ncomms10166
Jin, C. J., et al. (2016). British Journal of Nutrition, 116(10), 1682-1693. https://doi.org/10.1017/S0007114516004025
Kammoun, H. L., et. al. (2009). Journal of Clinical Investigation, 119(5), 1201-1215. https://doi.org/10.1172/JCI37007
Kasubuchi, M., et. al. (2015). Nutrients, 7(4), 2839-2849. https://doi.org/10.3390/nu7042839
Kelleher, R. J., et. al. (2017). Proceedings of the National Academy of Sciences of the United States of America, 114(4), 629-631. https://doi.org/10.1073/pnas.1619574114
Khan, S., et. al. (2020). Current Neuropharmacology, 18(11), 1106-1125. https://doi.org/10.2174/1570159x18666200528142429
Khanam, A., et. al. (2021). 10, 1097. https://doi.org/10.3390/cells
Kim, D. G., et. al. (2016). Journal of Neuroinflammation, 13(1). https://doi.org/10.1186/s12974-015-0467-5
Kitazawa, M., et al. Journal of Neuroscience, 25(39), 8843-8853. https://doi.org/10.1523/JNEUROSCI.2868-05.2005
Kivipelto, M., et. al. (2018). Nature Reviews Neurology, 14(11), 653-666. https://doi.org/10.1038/s41582-018-0070-3
Koeth, R. A., et. al. (2013). Nature Medicine, 19(5), 576-585. https://doi.org/10.1028/am.3145
Koga, S., et al. (2014). Neuroscience Letters, 571, 11-16. https://doi.org/10.1016/j.neulet.2014.04.028
Kolak, M., et. al. (2007). Diabetes, 56(8), 1960-1968. https://doi.org/10.2337/db07-0111
Koo, S. H. (2013). Clinical and molecular hepatology, 19(3), 210-215. https://doi.org/10.3350/cmh.2013.19.3.210
Lane, C. A., et. al. (2018). European Journal of Neurology, 25(1), 59-70. https://doi.org/10.1111/ene.13439
Leung, C., et al. (2016). Nature Reviews Gastroenterology and Hepatology, 13(7), 412-425. https://doi.org/10.1038/nrgastro.2016.85
Li, F., et al. (2013). Nature Communications, 4. https://doi.org/10.1038/ncomms3384
Li, N. M., et al. (2019). Neural Regeneration Research, 14(4), 658-665. https://doi.org/10.4103/1673-5374.247469
Li, X., et al. (2015). Clinical Interventions in Aging, 10, 549-560. https://doi.org/10.2147/CIA.S74042
Li, X. Y., et. al. (2020). Current Alzheimer Research, 16(14), 1254-1268. https://doi.org/10.2174/1567205017666200103111253
Liu, C. C., et. al. (2013). Nature Reviews Neurology, 9(2), 106-118. https://doi.org/10.1038/nmeurol.2012.263
Liu, R., et al. (2017). Nature Medicine, 23(7), 859-868. https://doi.org/10.1038/nm.4358
Liu, S., et al. (2020). https://dio.org/10_1007/812035-020-02073-3/
Liu, X., et al. (2024). Psychopharmacology. https://doi.org/10.1007/s00213-024-06530-y
Luukkonen, P. K., et al. (2016). http://www.r-project.org/
Lyn-Cook, L. E., et. al. (2009). Journal of Alzheimer's Disease, 16(4), 715-729. https://doi.org/10.3233/JAD-2009-0984
Ma, X., et. al (2012). J. Anim. Sci, 90, 266-268. https://doi.org/10.2527/jas50965
Mahley, R. W., et. al. (2000). www.annualreviews.org
Márquez-Aguirre, A. L., et. al. (2013). Food and Function, 4(8), 1237-1244. https://doi.org/10.1039/c3fo60083a
Masarone, M., et al. (2018). Oxidative Medicine and Cellular Longevity, 2018. https://doi.org/10.1155/2018/9547613
Masters, C. L., et. al. (2015). Nature Reviews Disease Primers, 1. https://doi.org/10.1038/nrdp.2015.56
Mayeux, R., et. al. (2012). Epidemiology of Alzheimer disease. Cold Spring Harbor Perspectives in Medicine, 2(8). https://doi.org/10.1101/cshperspect.a006239
Mehla, J., et. al. (2014). Journal of Alzheimer's Disease, 39(1), 145-162. https://doi.org/10.3233/JAD-131238
Mehlem, A., et. al. (2013). Nature Protocols, 8(6), 1149-1154. https://doi.org/10.1038/nprot.2013.055
Mellado-Mojica, E., et. al. (2017). Planta, 245(2), 265-281. https://doi.org/10.1007/s00425-016-2602-7
Mengr, A., et. al. (2023). Nutrients, 15(17). https://doi.org/10.3390/nu15173690
Ogretmen, B. (2017). Nature Reviews Cancer, 18(1), 33-50. https://doi.org/10.1038/nrc.2017.96
Oja, S. S., et. al. (2017). Neurochemical Research, 42(3), 713-720. https://doi.org/10.1007/s11064-016-2014-x
Padilla-Camberos, E., et al. (2018). Plant Foods for Human Nutrition, 73(1), 34-39. https://doi.org/10.1007/s11130-018-0654-5
Parnell, J. A., et. al. (2012). Liver International, 32(5), 701-711. https://doi.org/10.1111/j.1478-3231.2011.02730.x
Pilling, D., et al. (2021). American Journal of Pathology, 191(1), 131-143. https://doi.org/10.1016/j.ajpath.2020.09.011
Price, J. L., et al. (2001). Arch Neurol, http://archneur.jamanetwork.com/
Puglielli, L., et al. (2003). Journal of Biological Chemistry, 278(22), 19777-19783. https://doi.org/10.1074/jbc.M300466200
Ransom, B. R., et. al. (2012). Methods in Molecular Biology, 814, 3-7. https://doi.org/10.1007/978-1-61779-452-0_1
Reeves, P. G. (1997). J. Nutr, 127. https://academic.oup.com/jn/article-abstract/127/5/838S/4724089
Reitz, C., et. al. (2020). Neurology: Genetics, 6(5). https://doi.org/10.1212/NXG.0000000000000512
Rinella, M. E., et. al. (2016). Nature Reviews Gastroenterology and Hepatology, 13(4), 196-205. https://doi.org/10.1038/nrgastro.2016.3
Romero-Gómez, M., et. al. (2017). Journal of Hepatology, 67(4), 829-846. https://doi.org/10.1016/j.jhep.2017.05.016
Saif, M., et al. (2020). Nature Biomedical Engineering, 4(8), 801-813. https://doi.org/10.1038/s41551-020-0569-y
Salas-Salvadó, J., et. al. (2014). Diabetes Care, 34(1), 14-9. https://doi.org/10.2337/dc10-1288
Santos, C. Y., et. al. (2017). Alzheimer's and Dementia: Diagnosis, Assessment and Disease Monitoring, 7, 69-87. https://doi.org/10.1016/j.dadm.2017.01.005
Satoi, H., et. al. (2005). Neuroscience, 130(3), 657-666. https://doi.org/10.1016/j.neuroscience.2004.08.056
Scheltens, P., et. al. (2021). The Lancet, 397(10284), 1577-1590. https://doi.org/10.1016/S0140-6736(20) 32205-4
Segain, J.-P. (2000). Gut, 47.www.gutjnl.com
Sehgal, N., et. al. (2012). Proceedings of the National Academy of Sciences of the United States of America, 109(9), 3510-3515. https://doi.org/10.1073/pnas. 1112209109
Shah, R. (2013). Journal of the American Medical Directors Association, 14(6), 398-402. https://doi.org/10.1016/j.jamda.2013.01.014
Silva, M. V. F., et. al. (2019). Journal of Biomedical Science, 26(1). https://doi.org/10.1186/s12929-019-0524-x
Singh, B., et al. (2014a). Journal of Alzheimer's Disease, 39(2), 271-282. https://doi.org/10.3233/JAD-130830
Smith, C. J., et. al. (2019). Journal of Alzheimer's Disease, 68(3), 885-923. https://doi.org/10.3233/JAD-181089
Solah, V. A., et. al. (2006). Drug Metabolism and Disposition, 34(10), 1677-1687. https://doi.org/10.1124/dmd.106.010645
Sun, B., et al. (2019). British Journal of Nutrition, 122(4), 400-410. https://doi.org/10.1017/60007114519001399
Syeda, T., et. al. (2018). Journal of Alzheimer's Disease, 66(4), 1657-1682. https://doi.org/10.3233/JAD-180556
Tanabe, F., et al. (2013). Biochemical and Biophysical Research Communications, 441(1), 256-261. https://doi.org/10.1016/j.bbrc.2013.10.045
Tandra, S., et al. (2011). Journal of Hepatology, 55(3), 654-659. https://doi.org/10.1016/j.jhep.2010.11.021
Teixeira, V., et al. (2015). Cellular Signaling, 27(9), 1840-1849. https://doi.org/10.1016/j.cellsig.2015.06.001
Tomassini, B., et al. (2002). Mitochondria as sensors of sphingolipids.
Tong, M., et I. (2009). Journal of Alzheimer's Disease, 16(4), 705-714. https://doi.org/10.3233/JAD-2009-0983
Trinchese, F., et al. (2004). Annals of Neurology, 55(6), 801-814. https://doi.org/10.1002/ana.20101
Trovato, F. M. et. al. (2015). Clinical Nutrition, 34(1), 86-88. https://doi.org/10.1016/j.clnu.2014.01.018
Turnbaugh, P. J., et. al. (2006). Nature, 444(7122), 1027-1031. https://doi.org/10.1038/nature05414
Vandal, M., et al. (2014). Diabetes, 63, 4291-4301.
Vegas-Suárez, S., et al. (2022). Frontiers in Physiology, 13. https://doi.org/10.3389/fphys.2022.864263
Verkhratsky, A., et. al. (2010). Neurotherapeutics, 7, 399-412.
Vogt, N. M., et. al. (2005). Journal of Agricultural and Food Chemistry, 53(4), 1246-1253. https://doi.org/10.1021/jf048711u
Wang, F., et al. (2020). FEMS Microbiology Letters, 367(7). https://doi.org/10.1093/femsle/fnaa048
Wang, G., et. al. (2018). Advances in Biological Regulation, 70, 51-64. https://doi.org/10.1016/j.jbior.2018.09.013
Wang, G., et al. (2008). GLIA, 56(4), 449-456. https://doi.org/10.1002/glia.20626
Wang, X., et. al. (2019). Liver Research, 3(1), 25-30. https://doi.org/10.1016/j.livres.2019.01.006
Więckowska-Gacek, A., et. al. (2021). Ageing Research Reviews, 70. https://doi.org/10.1016/j.arr.2021.101397
Yang, Y., et al. (2013). PLOS ONE, 8(7). https://doi.org/10.1371/journal.pone.0069824
Zhang, L., et al. (2021). Trends in Endocrinology and Metabolism, 32(3), 159-169. https://doi.org/10.1016/j.tem.2020.12.003
Zhou, D., et. al. (2018). Experimental and Molecular Medicine, 50(12). https://doi.org/10.1038/s12276-018-0183-1
Zhou, H., et. al. (1998). Inhibition of Akt Kinase by Cell-permeable Ceramide and Its Implications for Ceramide-induced Apoptosis. http://www.jbc.org/

1. Agave fructans or agavins comprising a mixture of compounds having the formula: 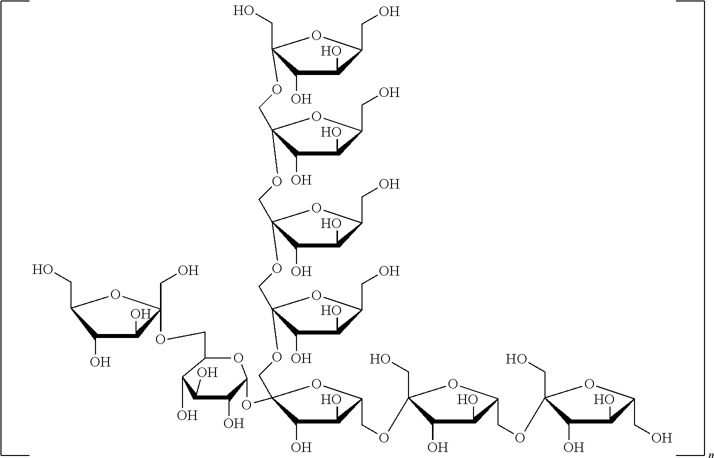
wherein n has a value of 2 to 32, which are effective for treating diseases related to neuroinflammation and intestinal dysbiosis.

2. Agave fructans or agavins in accordance to claim 1, wherein Agave fructanos are effective in the treatment of diseases and/or pathological conditions caused by aging, osteoporosis, obesity, and diabetes.

3. Agave fructans or agavins in accordance to claim 1, wherein the mixture thereof comes from Agave tequilana Jal., Agave tequilana Gto., Agave angustifolia Oax., Agave angustifolia Son., Agave potatorum Oax., Agave cantala Oax., Agave fourcroydes Yuc., Dasylirion spp. Chih or mixtures thereof.

4. Agave fructans or agavins in accordance to claim 1, wherein the diseases related to neuroinflammation comprising Alzheimer's disease, dementia, diseases and/or pathological conditions that negatively affect mental and/or cognitive functions, diseases and/or pathological conditions that cause degeneration of the central nervous system.

5. Agave fructans or agavins in accordance to claim 1, wherein the intestinal dysbiosis generates neuroinflammatory factors comprise short-chain fatty acids selected from the group comprising propionate and propionic acid, and said anti-neuroinflammatory factors comprise short-chain fatty acids selected from the group comprising acetate, butyrate, and butyric acid.

6. A pharmaceutical composition comprising a mixture of compounds comprising a mixture of Agave fructans or agavins comprising fructans from Agave tequilana Jal., Agave tequilana Gto., Agave angustifolia Oax., Agave angustifolia Son., Agave Potatorum Oax., Agave cantala Oax., Agave fourcroydes Yuc., Dasylirion spp. Chih. or mixtures thereof, for the treatment of diseases related to neuroinflammation, intestinal dysbiosis and/or pathological conditions caused by aging, osteoporosis, obesity and diabetes.

7. The pharmaceutical composition in accordance to claim 6, wherein said Agave fructans or agavins having the formula: 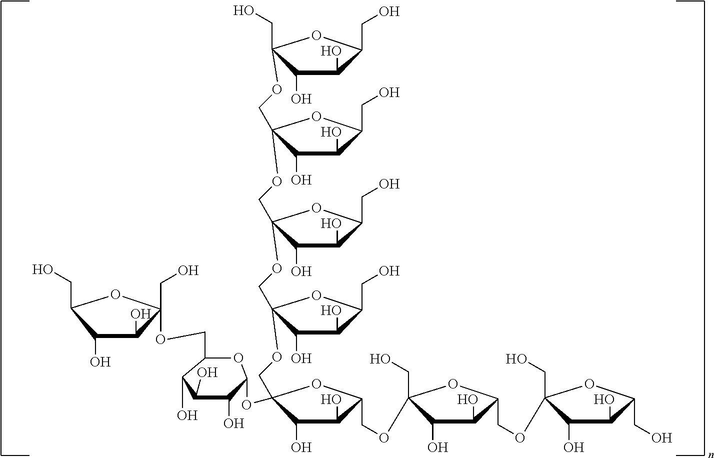
wherein n has a value of 2 to 32, which are effective for treating diseases related to neuroinflammation and intestinal dysbiosis.

8. The pharmaceutical composition in accordance to claim 6, wherein the diseases related to neuroinflammation comprising Alzheimer's disease, dementia, diseases and/or pathological conditions that negatively affect mental and/or cognitive functions, diseases and/or pathological conditions that cause degeneration of the central nervous system.

9. The pharmaceutical composition in accordance to claim 6, wherein the intestinal dysbiosis generates neuroinflammatory factors comprise short-chain fatty acids selected from the group comprising propionate and propionic acid, and said anti-neuroinflammatory factors comprise short-chain fatty acids selected from the group comprising acetate, butyrate, and butyric acid.

10. The pharmaceutical composition in accordance to claim 6, wherein said Agave fructanos or agavins are administered to the patient in a ratio of 3% to 20% w/w relative to the total weight of said composition.

11. The pharmaceutical composition according to claim 6, wherein said composition comprises a solid pharmaceutical form or a liquid pharmaceutical form, both of which comprise an pharmaceutical acceptable vehicle.

12. A food composition comprising a mixture of compounds comprising a mixture of Agave fructans or agavins comprising fructans from Agave tequilana Jal., Agave tequilana Gto., Agave angustifolia Oax., Agave angustifolia Son., Agave Potatorum Oax., Agave cantala Oax., Agave fourcroydes Yuc., Dasylirion spp. Chih. or mixtures thereof, for to stimulate the growth of probiotic bacteria (Coprococcus, Dehalobacterium and Moryella), modify the intestinal flora to reduce inflammation in subjects in need of it and treating diseases related with neuroinflammation and intestinal dysbiosis.

13. The food composition in accordance to claim 12, wherein said Agave fructans or agavins having the formula: 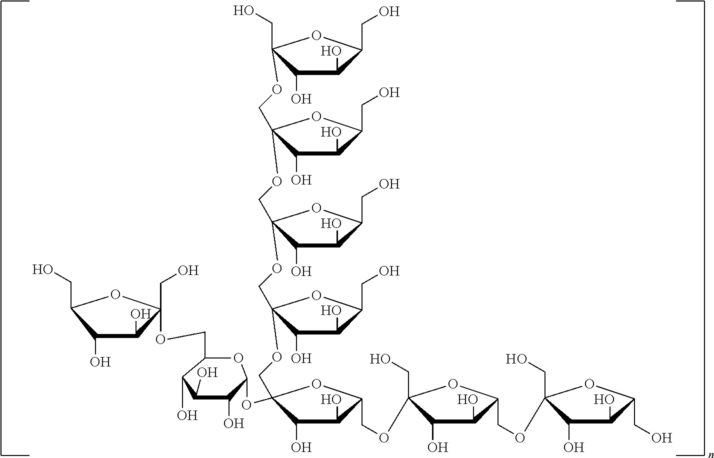
wherein n has a value of 2 to 32, which are effective in stimulating the growth of probiotic bacteria and modifying the intestinal flora to reduce inflammation in subjects who consume it.

14. The food composition in accordance to claim 12, wherein the diseases related to neuroinflammation comprising Alzheimer's disease, dementia, diseases and/or pathological conditions that negatively affect mental and/or cognitive functions, diseases and/or pathological conditions that cause degeneration of the central nervous system.

15. The food composition in accordance to claim 12, wherein said Agave fructanos or agavins are administered to the patient in a ratio of 3% to 20% w/w relative to the total weight of said composition.

16. The food composition according to claim 12, wherein said composition further comprises a mixture of food components such as casein, sucrose, cellulose, mineral mixture, vitamin mixture, I-cysteine, choline or tert-butylhydroquinone and an administration vehicle.

17. A method for the treatment of diseases caused by neuroinflammation or intestinal dysbiosis and/or pathological conditions caused by aging, osteoporosis, obesity and diabetes, wherein the method comprises the administration of Agave fructans or agavins of claim 1.

18. The treatment method in accordance to claim 17, wherein said Agave fructans or agavins having the formula: 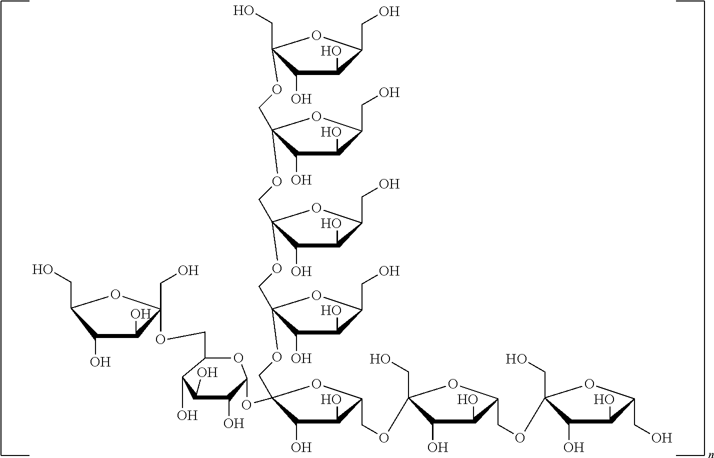
wherein n has a value of 2 to 32.

19. The treatment method in accordance to claim 17, wherein the Agave fructans or agavins comes from Agave tequilana Jal., Agave tequilana Gto., Agave angustifolia Oax., Agave angustifolia Son., Agave potatorum Oax., Agave cantala Oax., Agave fourcroydes Yuc., Dasylirion spp. Chih or mixtures thereof.

20. The treatment method in accordance to claim 17, wherein the diseases related to neuroinflammation comprising Alzheimer's disease, dementia, diseases and/or pathological conditions that negatively affect mental and/or cognitive functions, diseases and/or pathological conditions that cause degeneration of the central nervous system.

21. The treatment method in accordance to claim 17, wherein the intestinal dysbiosis generates neuroinflammatory factors comprise short-chain fatty acids selected from the group comprising propionate and propionic acid, and said anti-neuroinflammatory factors comprise short-chain fatty acids selected from the group comprising acetate, butyrate, and butyric acid.

22. The treatment method in accordance to claim 17, wherein said Agave fructans or agavins are administered to the patient in a ratio of 3% to 20% w/w relative to the total weight of said composition of claim 6.
